(19) United States
(12) Patent Application Publication
(10) Pub. No.: US 2024/0059709 A1
(43) Pub. Date: Feb. 22, 2024

(54) SYNTHESIS OF STRUCTURAL ANALOGS OF LARGAZOLE AND ASSOCIATED COMPOUNDS

(71) Applicants: COLORADO STATE UNIVERSITY RESEARCH FOUNDATION, Fort Collins, CO (US); CETYA THERAPEUTICS, INC., Fort Collins, CO (US)

(72) Inventors: Robert M. WILLIAMS, Ft. Collins, CO (US); Sivanagireddy KOTI, Ft. Collins, CO (US); Subhadip DE, Ft. Collins, CO (US); Anil M. SHELKE, Ft. Collins, CO (US); Ryan E. CERBONE, Ft. Collins, CO (US); Nobuyoshi YASUDA, Ft. Collins, CO (US)

(73) Assignees: COLORADO STATE UNIVERSITY RESEARCH FOUNDATION, Fort Collins, CO (US); CETYA THERAPEUTICS, INC., Fort Collins, CO (US)

(21) Appl. No.: 18/255,969

(22) PCT Filed: Dec. 16, 2021
(86) PCT No.: PCT/US2021/063719

§ 371 Date: Jun. 5, 2023

Related U.S. Application Data
(60) Provisional application No. 63/128,273, filed on Dec. 21, 2020.

(51) Int. Cl. C07D 513/18

(52) U.S. Cl. CPC C07D 513/18

(57) ABSTRACT

Disclosed are various synthetic methods to prepare structural analogs of largazole and derivatives thereof. One structural analog is an amide isostere of largazole. Another structural analog replaces the thiazole ring of largazole with a pyridine moiety or an oxazole moiety. Also disclosed are various intermediate compounds obtained when preparing structural analogs of

### CROSS-REFERENCE TO RELATED APPLICATIONS

This patent application claims the benefit of U.S. Provisional Patent Application No. 63/128,273, filed Dec. 21, 2020, which is incorporated herein by reference in its entirety for all purposes.

### STATEMENT REGARDING FEDERALLY SPONSORED RESEARCH OR DEVELOPMENT

This invention was made with Government support under Grant Number 2R42HL136068-02 awarded by the National Institutes of Health through the National Heart, Lung, and Blood Institute. The Government has certain rights in this invention.

### BACKGROUND OF INVENTION

Histone deacetylase (HDAC) inhibitors show great promise in the treatment of numerous forms of cancer and other conditions (see, e.g., Williams et al., WO 2016/144814, hereby incorporated by reference in its entirety). Various classes of HDAC inhibitors are known and generally fall into five categories: hydroxamates, benzamides, short-chain fatty acids, trifluoromethyloxadiazoles, and macrocycles. Specific examples of naturally occurring HDAC inhibitors include SAHA, trichostatin A, apicidin, trapoxin, azumamide, FK228, largazole, FR901375, and spiruchostatins. However, intense research efforts have focused on preparing structural analogs of these natural products in an attempt to understand the relationship between inhibitor structure and HDAC inhibition, to find more potent inhibitors, and to prepare inhibitors that have desired structural properties.

Particular interest has focused on the macrocycle largazole, as well as structural analogs thereof, including its amide isostere, in which the ester within the peptide ring is replaced with an amide functionality (see, e.g., Bowers et al., J. Am. Chem. Soc. 2009, 131, 2900-2905, and Pilon et al., Cancer Chemother Pharmacol. 2015, 75(4): 671-682, both of which are hereby incorporated herein by reference in their entireties). Largazole has been isolated as a natural product from the marine cyano-bacterium Symploca sp., and the total synthesis of largazole has been successfully completed by several research groups (see Ying et al., J. Am. Chem. Soc. 2008, 130, 8455-59, hereby incorporated by reference in its entirety). Largazole has several disadvantages in some circumstances, particularly its susceptibility to hydrolysis owing to its ester functionality within the macrocyclic ring, as well as conformational flexibility. The amide isostere of largazole addresses these disadvantages by being more stable to hydrolysis and having a more rigid structure, which affords increased HDAC binding, both of which are the result of replacing the ester functionality of largazole with an amide group.

Interest has also been focused on structural analogs of largazole, including those which retain the ester functionality in the macrocycle, but which vary other structural positions in the molecule. For example, in one structural analog of largazole, the thiazole ring in largazole can be replaced with a pyridine moiety or an oxazole moiety.

The known synthetic routes to the amide isosteres of largazole and other structural analogs thereof suffer from several drawbacks, including low yields, milligram scale production, complexity of synthesis, and high cost (see, e.g., Bowers et al.). Such known routes also pass through a macrocycle containing a reactive thiol group, such as the compound of formula (III), where R¹ is SH, which are highly biologically active, unstable, and difficult to handle. Therefore, there is a need for improved synthetic methods for the preparation of HDAC inhibitors, including improved methods for preparing structural analogs of largazole and derivatives thereof, as well as the chemical compounds prepared via these improved methods. The invention described herein is directed to these, as well as other, important needs.

### SUMMARY

Some benefits of the invention described herein include, employing synthetic methods that make use of macrocycles containing an alcohol functionality shown in formula (I), rather than a macrocycle having a thiol functionality. A macrocycle having an alcohol functionality is more stable, biologically inactive (see Table 1), and easier to handle (e.g., can be prepared in a crystalline form, see FIG. 10 XRD) than a comparable macrocycle with a thiol functionality. Other benefits of the methods disclosed herein include higher yields, more consistent yields, gram-scale production, easier and less complex synthesis, using less expensive materials, and reduced toxicity of intermediate compounds.

Disclosed is a method for preparing a compound of formula (I):

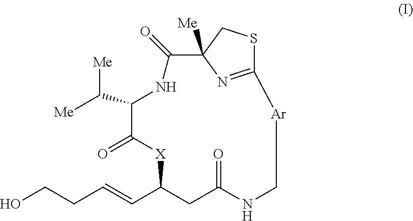

the method comprising reacting in one or more reactions: a compound of formula (IV), a compound of formula (V), and a compound of formula (VI), wherein the structures and variables are defined elsewhere herein.

Also disclosed is a method for preparing a compound of formula (III):

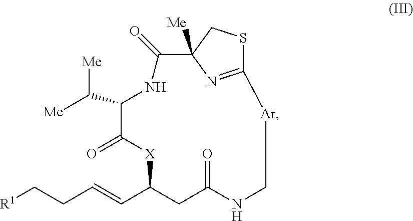

the method comprising reacting a compound of formula (I):

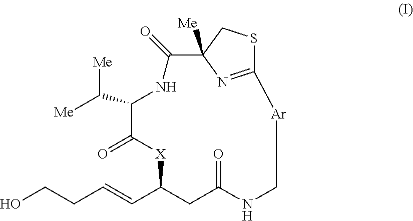

with an activating agent, thereby forming a compound of formula (II):

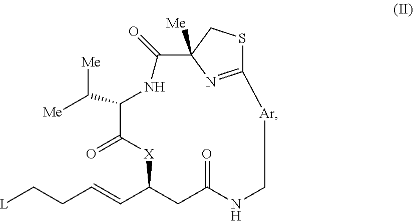

and reacting the compound of formula (II) with a nucleophile, thereby forming the compound of formula (III), wherein the structures, variables, activating agents, and nucleophile are as described elsewhere herein.

In some aspects, the compound of formula (I) is a compound of formula (I-a), (I-b), or (I-c):

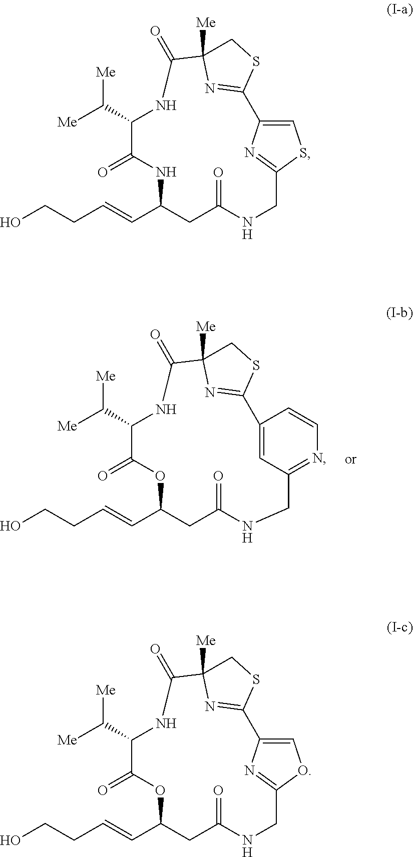 [SMILES: CC(C)[C@@H]1NC(=O)[C@]2(C)CSC(=N2)c2csc(n2)CNC(=O)C[C@@H](/C=C/CCO)NC1=O ; CC(C)[C@@H]1NC(=O)[C@]2(C)CSC(=N2)c2ccnc(c2)CNC(=O)C[C@@H](/C=C/CCO)OC1=O ; CC(C)[C@@H]1NC(=O)[C@]2(C)CSC(=N2)c2coc(n2)CNC(=O)C[C@@H](/C=C/CCO)OC1=O]

Additionally disclosed is a method for preparing a compound of formula (III):

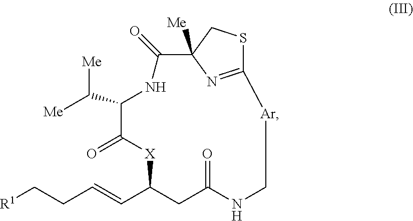

the method comprising reacting a compound of formula (II) with a nucleophile, wherein the structures, variables, activating agents, and nucleophile are as described elsewhere herein.

Also disclosed is a compound of formula (XXVI):

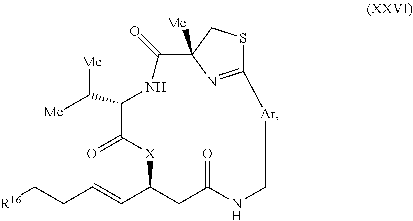

wherein the structures, variables, activating agents, and nucleophile are as described elsewhere herein.

In some aspects, the compound of formula (XXVI) is a compound of formula (I-a), (I-b), or (I-c):

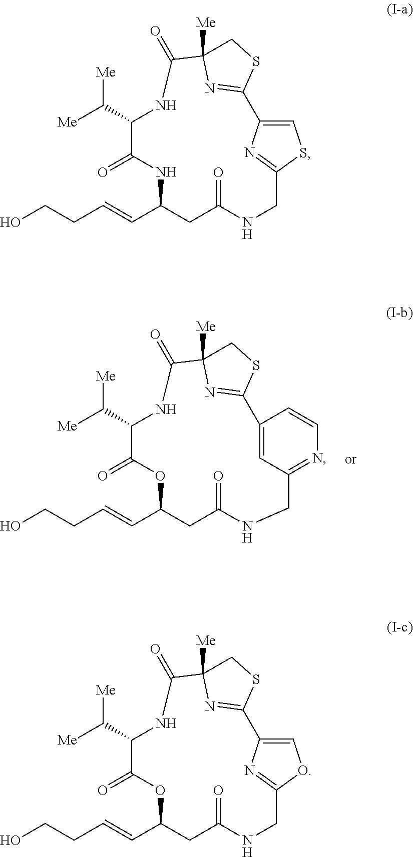 [SMILES: CC(C)[C@@H]1NC(=O)[C@]2(C)CSC(=N2)c2csc(n2)CNC(=O)C[C@@H](/C=C/CCO)NC1=O ; CC(C)[C@@H]1NC(=O)[C@]2(C)CSC(=N2)c2ccnc(c2)CNC(=O)C[C@@H](/C=C/CCO)OC1=O ; CC(C)[C@@H]1NC(=O)[C@]2(C)CSC(=N2)c2coc(n2)CNC(=O)C[C@@H](/C=C/CCO)OC1=O]

### BRIEF DESCRIPTION OF THE DRAWINGS

FIG. 1 depicts chemical reaction schemes, including Schemes 1, 2, and 3, that demonstrate certain methods and compounds disclosed herein.

FIG. 2 depicts chemical reaction schemes, including Schemes 4 and 5, that demonstrate certain methods and compounds disclosed herein.

FIG. 3 depicts a chemical reaction scheme, including Scheme 6, that demonstrate certain methods and compounds disclosed herein.

FIG. 4 depicts a chemical reaction scheme, including Scheme 7, that demonstrates certain methods and compounds disclosed herein.

FIG. 5 depicts a chemical reaction scheme, including Scheme 8, that demonstrates certain methods and compounds disclosed herein.

FIG. 6 depicts chemical reaction schemes, including Schemes 9 and 10, that demonstrate certain methods and compounds disclosed herein.

FIG. 7 depicts chemical reaction schemes, including Schemes 11 and 12, that demonstrate certain methods and compounds disclosed herein.

FIG. 8 depicts chemical reaction schemes, including Schemes 13, 14, and 15, that demonstrate certain methods and compounds disclosed herein.

FIG. 9 depicts chemical reaction schemes, including Schemes 16 and 17, that demonstrate certain methods and compounds disclosed herein.

FIG. 10 is an XRD of compound 10 (i.e., the compound of formula (I-a)).

### DETAILED DESCRIPTION

As used herein, a “suitable protecting group” is a chemical group that is used to temporarily block or protect a particular functional moiety, e.g., COOH, O, S, or N, on a molecule so that a reaction can be carried out selectively at another reactive site on the molecule. A suitable protecting group typically reacts with a COOH, O, S, or N functionality in acceptable yield to give a protected group that is stable to the projected reactions. A suitable protecting group typically should be selectively removable in acceptable yield by one or more reagents that do not react with other portions of the molecule, unless such reactions are desired. As detailed herein, a variety of suitable protecting groups may be employed to protect carboxylic acid, oxygen, sulfur, and nitrogen functionalities, and such protecting groups are termed herein, respectively, as “suitable carboxylic acid protecting groups,” “suitable alcohol protecting groups,” “suitable thiol protecting groups,” and “suitable amine protecting groups.” Such suitable protecting groups are well-known in the art and include those described in several published sources, including, for example, (1) Protective Groups in Organic Synthesis, Theodora W. Greene and Peter G. M. Wuts, 2006, published by John Wiley and Sons; and (2) Bioconjugate Techniques, Greg T. Hermanson, 2013, published by Academic Press; both of which are hereby incorporated by reference in their entireties.

“Suitable alcohol protecting groups” are well known in the art and include, for example, tert-butyldimethylsilyl (TBDMS or TBS), tert-butyldiphenylsilyl (TBDPS), or tetrahydropyran (THP).

“Suitable thiol protecting groups” are well known in the art and include, for example, triphenylmethyl (Trt or trityl).

“Suitable amine protecting groups” are well known in the art and include, for example, 9-fluorenylmethoxy carbamate (Fmoc), t-butyl carbamate (Boc), or benzyloxy carbamate (Cbz).

“Suitable carboxylic acid protecting groups” are well known in the art and include, for example, C₁-C₆ linear or branched alkyl, C₁-C₆ linear or branched alkyl substituted with one or more aryl or heteroaryl in any position, or alkylsilyl. In some aspects, the C₁-C₆ linear or branched alkyl is, for example, methyl, ethyl, propyl, isopropyl, allyl, butyl, 2-butyl, tert-butyl, benzyl, or substituted benzyl, each of which can be substituted with one or more aryl or heteroaryl in any position. In some aspects, the alkylsilyl is, for example, 2-(trimethylsilyl)ethyl.

In some aspects, any carboxylic, amine, or thiocarboxylic functionality on any compound disclosed herein can be present, e.g., as H, an alkali metal (or ion), an alkaline earth metal (or ion), a salt, or any combination thereof.

When a number of carbon atoms of a functional or other group (e.g., alkyl group) is specified herein, it is intended that the number refers to the exact number of carbon atoms, or range of carbon atoms, that is specified. For example, if an alkyl is specified to contain 4 to 8 carbon atoms, an alkyl group containing 12 carbon atoms would not qualify; rather, only an alkyl group that contains 4, 5, 6, 7, or 8 carbon atoms would qualify. In addition, when a number of carbon atoms is specified as a range, it is intended that any sub-range or individual number of carbon atoms falling within the indicated range are specifically contemplated, as if each such sub-range or individual carbon atom numbers are explicitly set forth herein. For example, a C₁-C₂₄ carbon atom range is intended to include 1, 2, 3, 4, 5, 6, 7, 8, 9, 10, 11, 12, 13, 14, 15, 16, 17, 18, 19, 20, 21, 22, 23, or 24 carbon atoms. In addition, all sub-ranges that can be made therefrom are also specifically contemplated, including, but not limited to, 1-2, 1-3, 1-4, 1-5, 1-6, 1-7, 1-8, 1-9, 1-10, 1-12, 1-14, 1-16, 1-18, 1-20, 1-22, 1-24, 2-3, 2-4, 2-5, 2-6, 2-7, 2-8, 2-9, 2-10, 2-12, 2-14, 2-16, 2-18, 2-20, 2-22, 2-24, 3-4, 3-5, 3-6, 3-7, 3-8, 3-9, 3-10, 3-12, 3-14, 3-18, 3-24, 4-5, 4-8, 4-10, 4-12, 4-16, 4-20, 4-24, 6-8, 6-10, 6-12, 6-16, 6-20, 6-24, 8-10, 8-12, 8-16, 8-20, 8-24, 10-14, 10-20, 10-24, 12-18, or 12-24 carbon atoms.

In performing the various reaction steps disclosed herein, any suitable reaction conditions may be used. Such reaction conditions may include a solvent, an acid, a base, a reagent, a catalyst, a time, a temperature, a pH, or any combination thereof. In some aspects, certain reaction products may be purified before proceeding to the next step, or certain reaction products may be carried forward crude to the next reaction step. The various reaction conditions and synthetic reactions disclosed herein are exemplary, but any suitable reaction conditions or synthetic reactions can be employed such as, for example, those disclosed in Advanced Organic Chemistry—Part B: Reactions and Synthesis, Francis A. Carey and Richard J. Sundberg, 4^{th} Edition, 2001, published by Springer, hereby incorporated by reference in its entirety.

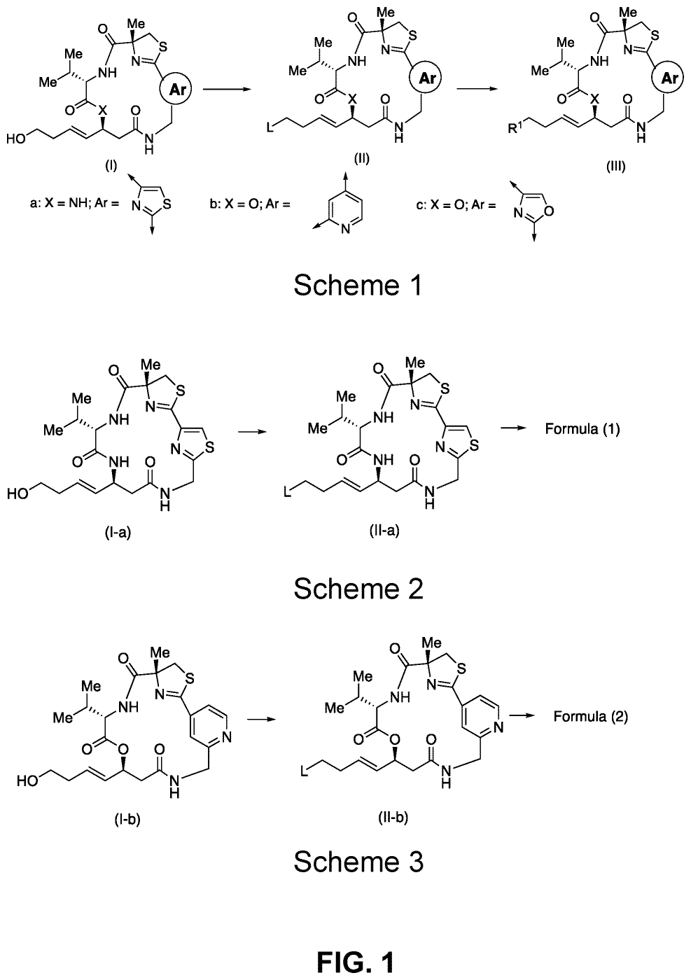

As shown in Scheme 1 in FIG. 1, disclosed herein is a method for preparing a compound of formula (III):

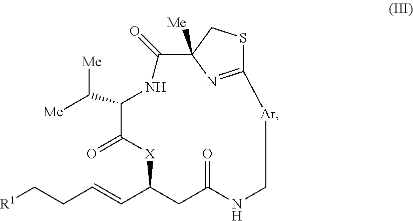

the method comprising:
reacting a compound of formula (I):

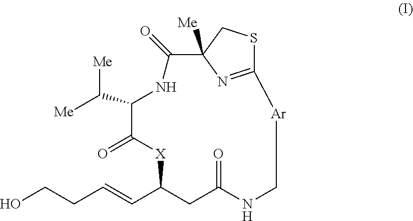

with an activating agent selected from C₁-C₂₀ linear or branched alkyl-sulfonyl halide, substituted alkyl-sulfonyl halide, arylsulfonyl halide, substituted arylsulfonyl halide, halo-phosphite (e.g., chlorophosphite), or a halogenating reagent, thereby forming a compound of formula (II):

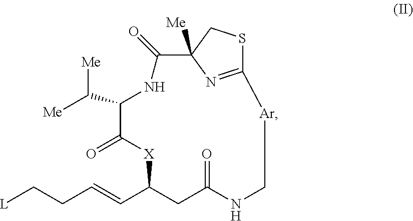

and reacting the compound of formula (II) with a nucleophile comprising an R¹ group, thereby forming the compound of formula (III).
wherein:
X is O or NH;
Ar is thiazolyl, pyridinyl, or oxazolyl;
L is a leaving group selected from C₁-C₂₄ linear or branched carbon sulfonate, substituted C₁-C₂₄ linear or branched sulfonate, arylsulfonate, substituted arylsulfonate, heteroarylsulfonate, substituted heteroarylsulfonate, phosphate, or halogen;
R¹ is R₂COS, R₃S, or R⁴R₅N, and
R², R³, R⁴, and R⁵independently are hydrogen, C₁₋₂₄ linear alkyl (e.g., methyl, ethyl, n-propyl, n-butyl, n-pentyl, n-hexyl, n-heptyl, n-octyl and so forth), C₁-C₂₄ linear alkyl substituted with one or more aryl or heteroaryl in any position (e.g., substituted with phenyl, pyridinyl, furanyl, pyrrolyl, thiophenyl, and so forth, or any combination thereof), C₁-C₂₄ branched alkyl (e.g., isopropyl, isobutyl, sec-butyl, tert-butyl, and so forth), C₁-C₂₄ branched alkyl substituted with one or more aryl or heteroaryl in any position (e.g., isopropyl substituted with phenyl, isobutyl substituted with pyridinyl, and so forth), C₃-C₂₄ cyclic alkyl (e.g., cylopropyl, cyclobutyl, cyclopentyl, cyclohexyl, methylcyclopropyl, and so forth), C₃-C₂₄ cyclic alkyl substituted with one or more aryl or heteroaryl in any position (e.g., cyclohexyl substituted with phenyl, and so forth), C₁-C₂₄ alkenyl, C₁-C₂₄ alkynyl, aryl (e.g., naphthalenyl, phenyl, pryridinyl, furanyl, pyrrolyl, thiophenyl, and so forth), heteroaryl (e.g., pryridinyl, furanyl, pyrrolyl, thiophenyl, and so forth), diphenyl methyl, triphenylmethyl, or any substituted version thereof. In some aspects, R¹ is R₂COS and R² is n-heptyl.

Also disclosed herein as shown in Scheme 1 in FIG. 1 is a method for preparing a compound of formula (III) comprising reacting the compound of formula (II) with a nucleophile comprising an R¹ group, wherein the variables in the compounds of formula (II) and (III) are as defined elsewhere herein. In other words, in some aspects, a method for preparing the compound of formula (III) starts from the compound of formula (II) rather than the compound of formula (I). However, in other aspects, a method for preparing the compound of formula (III) starts from the compound of formula (I) and passes through the compound of formula (II), as disclosed elsewhere herein.

In some aspects, L is any suitable leaving group. The term “leaving group” is well-known in the art, but, solely for illustration, a leaving group is a molecular fragment whose bond to an underlying molecule is broken, and the leaving group departs from the molecule with the pair of electrons from the broken bond. In a substitution reaction, a nucleophile typically forms a bond to the position on the underlying molecule where the leaving group was previously located. Suitable leaving groups include, for example, C₁-C₂₄ linear or branched carbon sulfonate, substituted C₁-C₂₄ linear or branched sulfonate (e.g., substituted with any group, such as alkyl, alkenyl, alkynyl, aryl, heteroaryl, heterocycle, halide, hydroxyl, alkoxyl, and so forth, or any combination thereof), arylsulfonate, substituted arylsulfonate (e.g., substituted with any group, such as alkyl, alkenyl, alkynyl, aryl, heteroaryl, heterocycle, halide, hydroxyl, alkoxyl, and so forth, or any combination thereof), heteroarylsulfonate, substituted heteroarylsulfonate ( e.g., substituted with any group, such as alkyl, alkenyl, alkynyl, aryl, heteroaryl, heterocycle, halide, hydroxyl, alkoxyl, and so forth, or any combination thereof), phosphate, or halogen. For example, in some aspects, L is OMs, OTs, OTf, 2,2,2-trifluoroethanesulfonate, n-octanesulfonate, benzenesulfonate, OP(═O)(OMe)₂, OP(═O)(OEt)₂, OP(═O)(OPh)₂, I, Br, Cl, or F.

In some aspects, as shown in Scheme 1 in FIG. 1, disclosed herein is a method comprising producing the compound of formula (II) from a compound of formula (I):

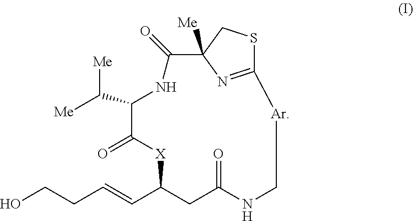

In some aspects, disclosed herein is a method comprising producing the compound of formula (II) from a compound of formula (IX):

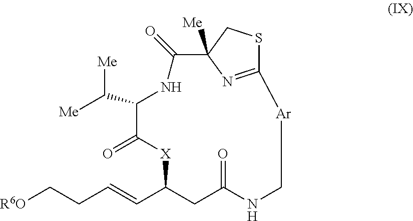

by converting OR⁶ of the compound of formula (IX) into the leaving group L; wherein Re is H or a suitable alcohol protecting group.

In some aspects, converting ORe into the leaving group L comprises: when Re is the suitable alcohol protecting group, deprotecting the suitable alcohol protecting group to provide the compound of formula (IX) where R⁶ is H; and reacting the compound of formula (IX) where Re is H with an activating agent selected from C₁-C₂₀ linear or branched alkyl-sulfonyl halide, substituted alkyl-sulfonyl halide, arylsulfonyl halide, substituted arylsulfonyl halide, halo-phosphite (e.g., chlorophosphite), or a halogenating reagent. Suitable specific activating agents are disclosed elsewhere herein.

In some aspects, the compound of formula (IX) is a compound of formula (I):

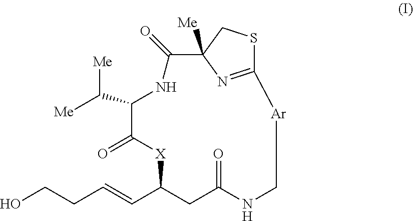

and the method comprises reacting the compound of formula (I) with an activating agent selected from C₁-C₂₀ linear or branched alkyl-sulfonyl halide, substituted alkyl-sulfonyl halide, arylsulfonyl halide, substituted arylsulfonyl halide, halo-phosphite (e.g., chlorophosphite), or a halogenating reagent, thereby forming the compound of formula (II).

In some aspects, the compound of formula (I) or the compound of formula (IX) is a compound of formula (I-a), (I-b), or (I-c):

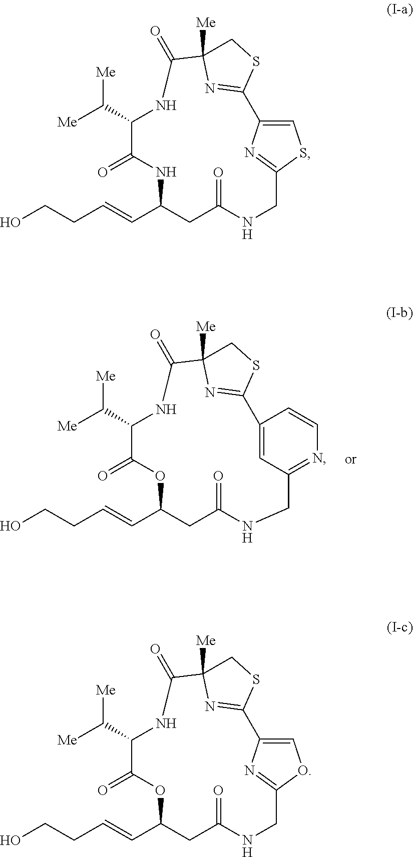 [SMILES: CC(C)[C@@H]1NC(=O)[C@]2(C)CSC(=N2)c2csc(n2)CNC(=O)C[C@@H](/C=C/CCO)NC1=O ; CC(C)[C@@H]1NC(=O)[C@]2(C)CSC(=N2)c2ccnc(c2)CNC(=O)C[C@@H](/C=C/CCO)OC1=O ; CC(C)[C@@H]1NC(=O)[C@]2(C)CSC(=N2)c2coc(n2)CNC(=O)C[C@@H](/C=C/CCO)OC1=O]

In some aspects, Ar is:

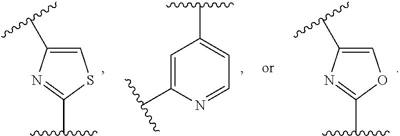 [SMILES: *c1csc(*)n1 ; *c1ccnc(*)c1 ; *c1coc(*)n1]

In some aspects, the nucleophile employed in the method is thiooctanoic acid, such that the compound of formula (III) is a compound of formula (XXV):

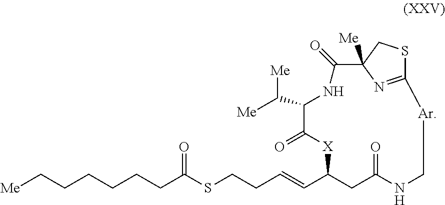

In some aspects, the nucleophile employed in the method is thiooctanoic acid, X is NH, and Ar is

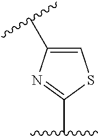 [SMILES: *c1csc(*)n1]

such that the compound of formula (III) is a compound of formula (1):

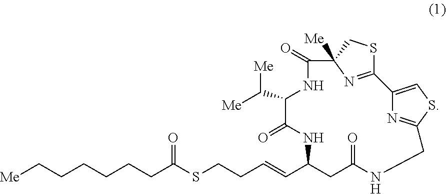 [SMILES: CCCCCCCC(=O)SCC/C=C/[C@@H]1CC(=O)NCc2nc(cs2)C2=N[C@@](C)(CS2)C(=O)N[C@@H](C(C)C)C(=O)N1]

In some aspects, disclosed herein is a method for preparing a compound of formula (1, III-a), depicted in Scheme 2 of FIG. 1:

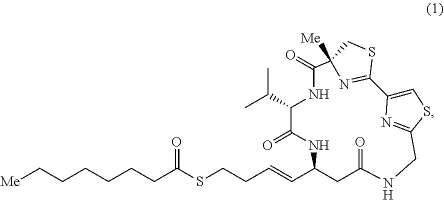 [SMILES: CCCCCCCC(=O)SCC/C=C/[C@@H]1CC(=O)NCc2nc(cs2)C2=N[C@@](C)(CS2)C(=O)N[C@@H](C(C)C)C(=O)N1]

the method comprising producing the compound of formula (1) from a compound of formula (I-a) via formula (II-a), in which L is as defined elsewhere herein.

In some aspects, the nucleophile employed in the method is thiooctanoic acid, X is O, and Ar is

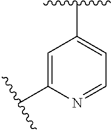 [SMILES: *c1ccnc(*)c1]

such that the compound of formula (III) is a compound of formula (2):

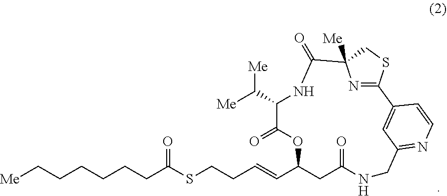 [SMILES: CCCCCCCC(=O)SCC/C=C/[C@@H]1CC(=O)NCc2cc(ccn2)C2=N[C@@](C)(CS2)C(=O)N[C@@H](C(C)C)C(=O)O1]

In some aspects, disclosed herein is a method for preparing a compound of formula (2, III-b), depicted in Scheme 3 of FIG. 1:

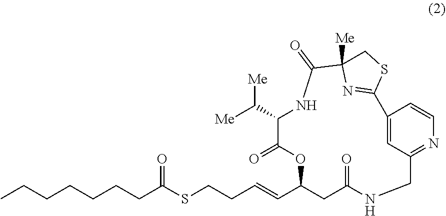 [SMILES: CCCCCCCC(=O)SCC/C=C/[C@@H]1CC(=O)NCc2cc(ccn2)C2=N[C@@](C)(CS2)C(=O)N[C@@H](C(C)C)C(=O)O1]

the method comprising producing the compound of formula (2) from a compound of formula (I-b) via formula (II-b), where L is a leaving group as defined elsewhere herein.

In some aspects, the nucleophile employed in the method is thiooctanoic acid, X is O, and Ar is

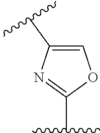 [SMILES: *c1coc(*)n1]

such that the compound of formula (III) is a compound of formula (3):

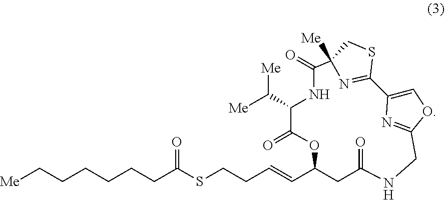 [SMILES: CCCCCCCC(=O)SCC/C=C/[C@@H]1CC(=O)NCc2nc(co2)C2=N[C@@](C)(CS2)C(=O)N[C@@H](C(C)C)C(=O)O1]

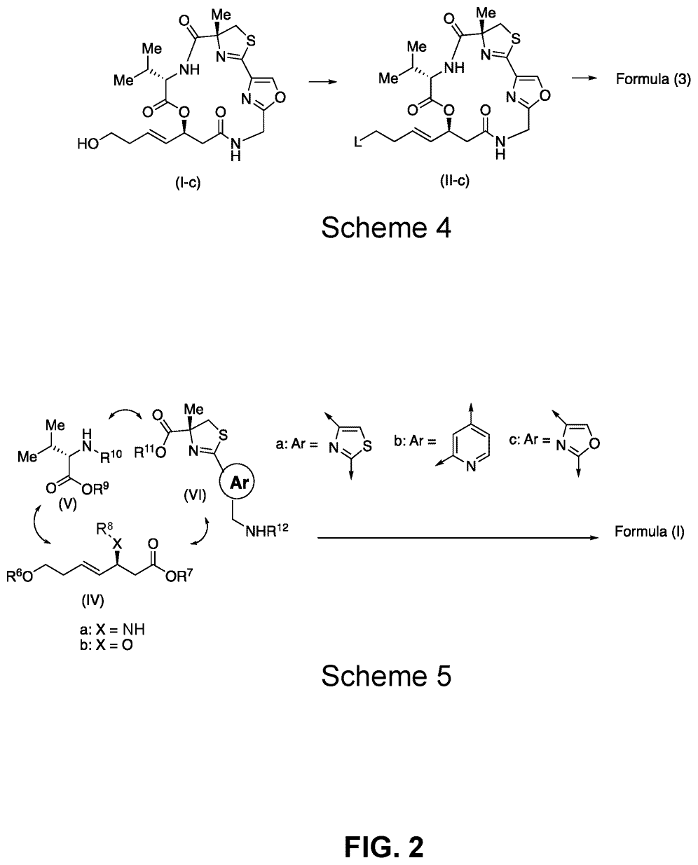

In some aspects, disclosed herein is a method for preparing a compound of formula (3, III-c), depicted in Scheme 4 of FIG. 2:

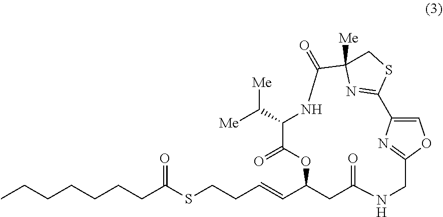 [SMILES: CCCCCCCC(=O)SCC/C=C/[C@@H]1CC(=O)NCc2nc(co2)C2=N[C@@](C)(CS2)C(=O)N[C@@H](C(C)C)C(=O)O1]

the method comprising producing the compound of formula (3) from a compound of formula (I-c) via formula (II-c), where L is a leaving group as defined elsewhere herein.

In some aspects, conversion of a compound of formula (I) to a compound of formula (II) with suitable activating reagent and suitable base in suitable solvent can be achieved at a temperature of −78 to 120° C., −50 to 80° C, −20 to 50° C., or −10 to 10° C.

In some aspects, suitable activating reagents include, but are not limited to, C₁-C₂₀ linear or branched alkyl-sulfonyl halide, substituted alkyl-sulfonyl halide, arylsulfonyl halide, substituted arylsulfonyl halide, halo-phosphite (e.g., chlorophosphite), halogenating reagents, mesyl chloride, tosyl chloride, trifluoromethanesulfonyl chloride, 2,2,2-trifluoroethanesulfonyl chloride, benzenesulfonyl chloride, n-octanesulfonyl chloride, or any combination thereof.

In some aspects, suitable bases include, but are not limited to, organic bases such as triethylamine, diisopropylethylamine, pyridine, lutidine, collidine, DBU, DABCO, and inorganic bases such as NaHCO₃, KHCO₃, Na₂CO₃, K₂CO₃, CS₂CO₃, NaOH, KOH, or any combination thereof.

In some aspects, suitable solvents include, but are not limited to, hydrocarbon, toluene, chlorobenzene, isopropyl acetate, ethyl acetate, methyl acetate, diethyl ether, CPME, MTBE, THF, 2-methyl-THF, dioxane, acetone, methyl ethyl ketone, acetonitrile, DMF, DMAC, DMSO, DCM, water, or any combination thereof

In some aspects, conversion of a compound of formula (II) to a compound of formula (III) with suitable nucleophile in a suitable solvent can be achieved at a temperature of −20 to 70° C., −10 to 60° C., 0 to 50° C., or 0 to 30° C.

In some aspects, suitable solvents include, but are not limited to, hydrocarbon, toluene, chlorobenzene, isopropyl acetate, ethyl acetate, methyl acetate, diethyl ether, CPME, MTBE, THF, 2-methyl-THF, dioxane, acetone, methyl ethyl ketone, acetonitrile, DMF, DMAc, DMSO, DCM, water, or any combination thereof.

In some aspects, suitable nucleophiles include, but are not limited to, R₂COS⁻, R₃S⁻, or R₄R₅N⁻; R², R³, R⁴, R⁵are defined elsewhere herein. In some aspects, the nucleophile is n-C₇H₁₅COS⁻.

In some aspects, disclosed herein is a method for the preparation of a compound of formula (I), depicted in Scheme 5 of FIG. 2. In some aspects, the method comprises producing the compound of formula (I) from a compound of formula (IV; beta-functioned ester), a compound of formula (V), and a compound of formula (VI). The compound of formula (I) can be prepared using standard ester formation and amide formation reactions with a suitable protecting group on nitrogen and/or oxygen atoms. The order of bond formation between three components (IV, V, and VI) generally is not important, and thus any order of bond formation is feasible. In Scheme 5 of FIG. 2, R⁶ is H or a suitable alcohol protecting group; R⁷, R⁹, and R¹¹ independently are H, an alkali metal ion, an alkaline earth metal ion, or a suitable carboxylic acid protecting group, such as, for example, C₁-C₆ alkyl group, substituted C₁-C₆ alkyl group, 2-trimethylsilylethyl, benzyl, or substituted benzyl; R⁸, R¹⁰ and R¹² are independently, H, or a suitable nitrogen protecting group. Ar is thiazole, pyridine, or oxazole. X is O or NH. In some aspects, any N atom is a quaternary ammonium.

In some aspects, the method further comprises producing the compound of formula (I):

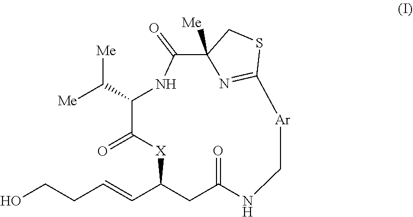

or the compound of formula (IX):

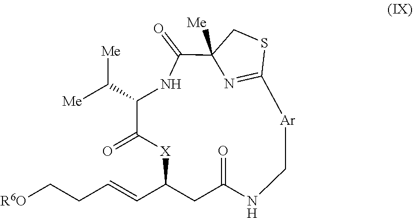

from one or more reactions comprising:
a compound of formula (IV):

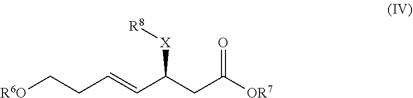

a compound of formula (V):

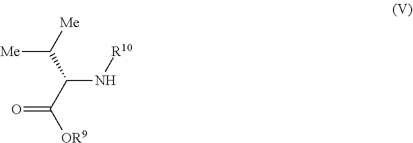

and a compound of formula (VI):

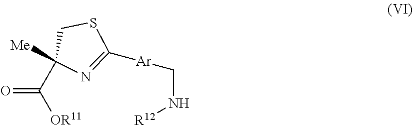

wherein:
R⁸ is H or a suitable alcohol protecting group;
R⁸, R¹⁰, and R¹² independently are H, a suitable amine protecting group, or a chiral group;
R⁷, R⁹, and R¹¹ independently are H, an alkali metal ion, an alkaline earth metal ion, or a suitable carboxylic acid protecting group;
optionally wherein the suitable carboxylic acid protecting group is selected from C₁-C₆ linear or branched alkyl, C₁-C₆ linear or branched alkyl substituted with one or more aryl or heteroaryl in any position, or alkylsilyl;
optionally wherein the C₁-C₆ linear or branched alkyl, or the C₁-C₆ linear or branched alkyl substituted with one or more aryl or heteroaryl in any position, is selected from methyl, ethyl, propyl, isopropyl, allyl, butyl, 2-butyl, tert-butyl, benzyl, or substituted benzyl;
optionally wherein the alkylsilyl is 2-(trimethylsilyl)ethyl; and
optionally wherein any N atom is a quaternary ammonium.

In some aspects, the compound of formula (I) or the compound of formula (IX) is a compound of formula (I-a), (I-b), or (I-c):

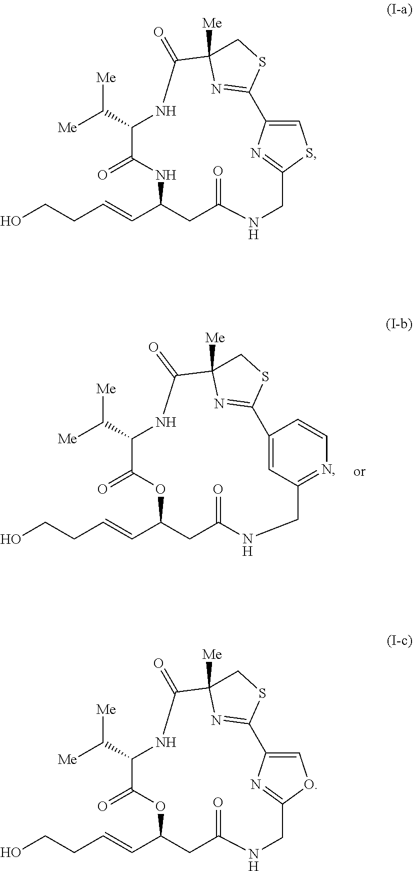 [SMILES: CC(C)[C@@H]1NC(=O)[C@]2(C)CSC(=N2)c2csc(n2)CNC(=O)C[C@@H](/C=C/CCO)NC1=O ; CC(C)[C@@H]1NC(=O)[C@]2(C)CSC(=N2)c2ccnc(c2)CNC(=O)C[C@@H](/C=C/CCO)OC1=O ; CC(C)[C@@H]1NC(=O)[C@]2(C)CSC(=N2)c2coc(n2)CNC(=O)C[C@@H](/C=C/CCO)OC1=O]

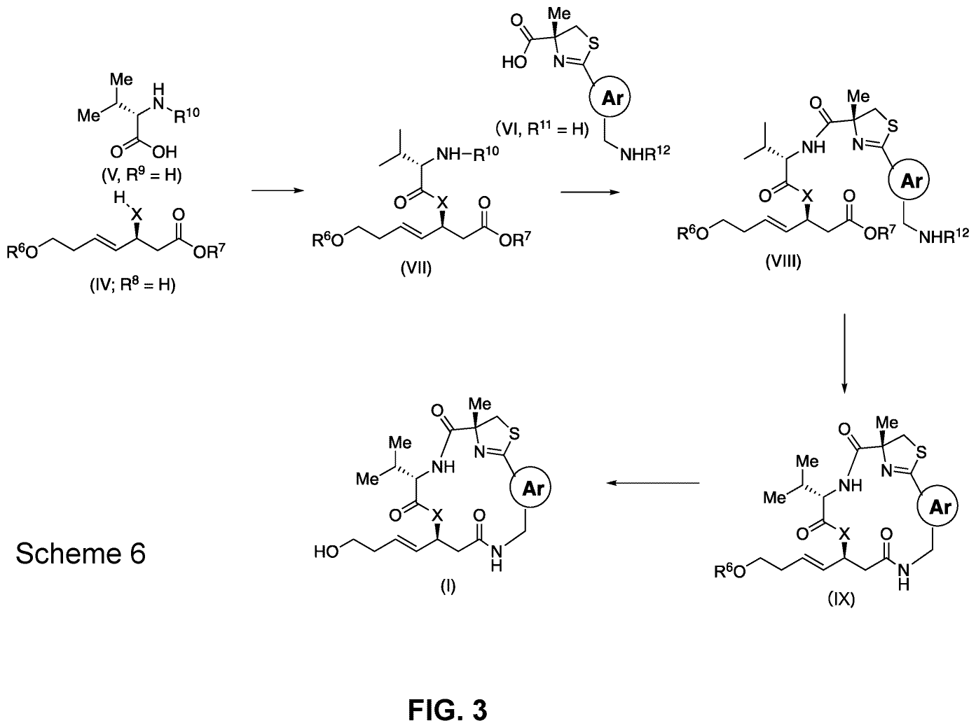

In some aspects, the compound of formula (I) is produced by a method depicted in Scheme 6 of FIG. 3, comprising:
reacting via condensation of a compound of formula (IV, R⁸=H) with a compound of formula (V, R⁹=H) to form a compound of formula (VII):
reacting the compound of formula (VII, R¹⁰=H) with a compound of formula (VI, R¹¹=H) under amidation conditions to form a compound of formula (VIII);
reacting the compound of formula (VIII, R⁷ and R¹¹=H) under amidation conditions to from a macrocyclic compound of formula (IX);
reacting the compound of formula (IX) under deprotection conditions to remove the alcohol protecting group to from a compound of formula (I).

In some aspects, the compound of formula (IV) is reacted with the compound of formula (V) via condensation to form a compound of formula (VII):

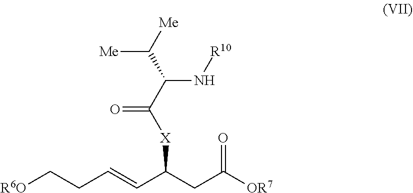

the compound of formula (VII) is reacted with the compound of formula (VI) via amidation to form a compound of formula (VIII):

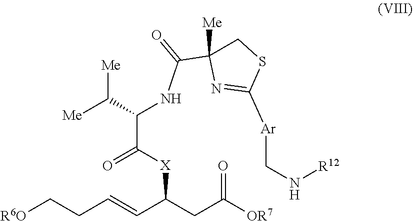

and the compound of formula (VIII) is reacted by amidating ring closure to form the compound of formula (IX), in which R⁶, R⁷, R¹⁰, and R¹² are as defined elsewhere herein. In some aspects, any N atom is a quaternary ammonium.

In some aspects, R⁶ is H or a suitable alcohol protecting group; and
in reacting the compound of formula (IV) with the compound of formula (V): R⁸ is H, and when X is N, the N is neutral or positively charged; R⁷ is a suitable carboxylic acid protecting group; R¹⁰ is a suitable amine protecting group; and R⁹ is H, an alkali metal ion, or an alkaline earth metal ion;
in reacting the compound of formula (VI) with the compound of formula (VII): R⁷ is a suitable carboxylic acid protecting group; R¹⁰ is H, and optionally R¹⁰ is attached to a positively charged nitrogen atom; R¹¹ is H, an alkali metal ion, or an alkaline earth metal ion; and R¹² is a suitable amine protecting group; and
in reacting the compound of formula (VIII) by amidating ring closure: R⁸ is H, an alkali metal ion, or an alkaline earth metal ion, and when X is N, the N is neutral or positively charged; and R¹² is H, and optionally R¹² is attached to a positively charged nitrogen atom.

In some aspects, selection of a protecting group is properly chosen based on orthogonality with other protecting groups. Suitable methods for removal of protecting groups for a given functional group depends on the nature of protecting groups in the same molecule.

In some aspects, suitable preferred nitrogen protecting groups are Boc or Fmoc. Those protecting groups can be selectively removed by acidic conditions or basic conditions.

In some aspects, suitable carboxylic acid protecting groups are C₁-C₆ alkyl group, substituted C₁-C₆ alkyl group, 2-trimethylsilylethyl, benzyl, or substituted benzyl. In some aspects, suitable carboxylic acid protecting groups are methyl, ethyl, or 2-trimethylsilylethyl. Such protecting groups can be selectively removed by basic conditions and acidic conditions.

In some aspects, condensation reagents are used to prepare an ester bond or an amide bond from carboxylic acids and alcohols or amines. In some aspects, condensation reagents include HATU, EDC, DCC, T3P, HBTU, TATU, TBTU, COMU, HOTU, BOP, PyBOP, and PyBroP.

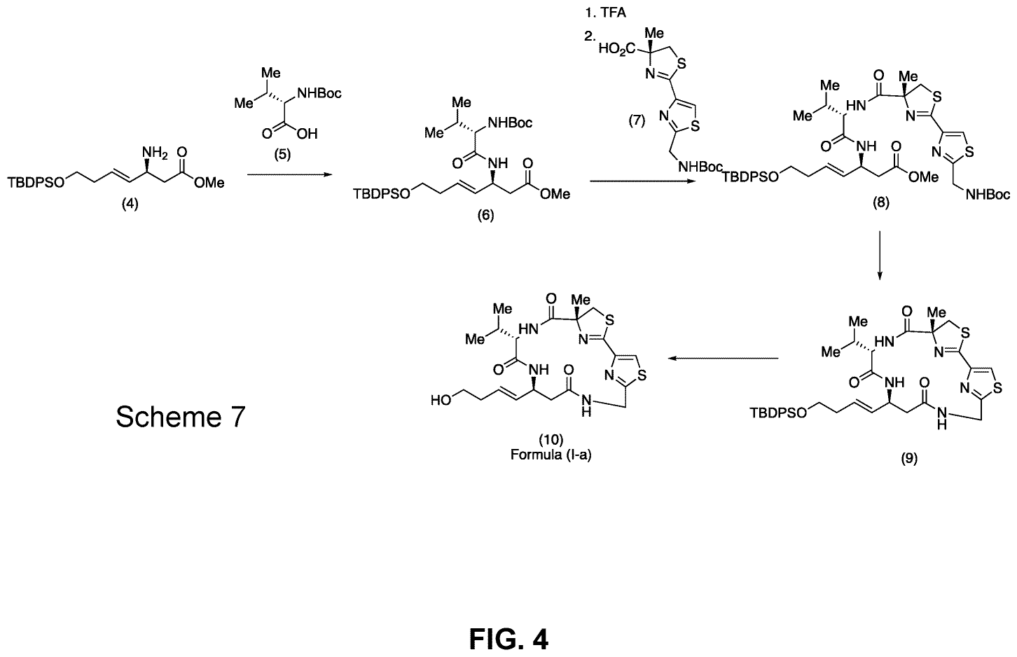

In some aspects, the compound of formula (I-a) is prepared as depicted in Scheme 7 of FIG. 4. Protecting groups and reaction conditions are the same as described elsewhere herein for the same or similarly structured compounds. The protecting groups and reaction conditions depicted in Scheme 7 are merely exemplary and not to be construed as limiting in any way. Compound 7 has been reported in references 1-3, which are hereby incorporated by reference in their entireties for all purposes.

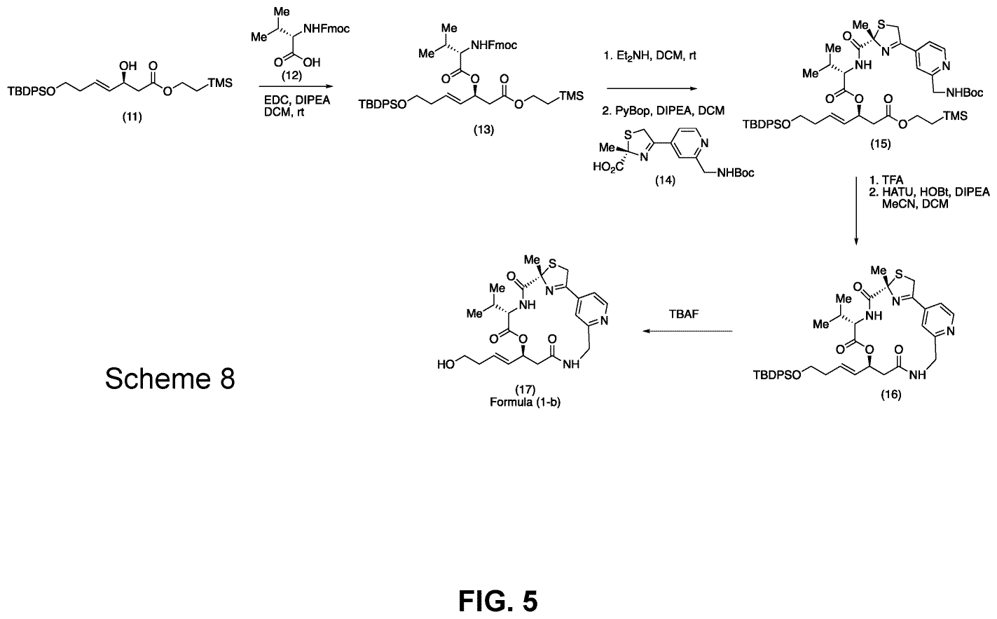

In some aspects, the compound of formula (I-b) is prepared as depicted in Scheme 8 in FIG. 5. The protecting groups and reaction conditions are the same as described elsewhere herein for the same or similarly structured compounds. The protecting groups and reaction conditions depicted in Scheme 8 are merely exemplary and not to be construed as limiting in any way. Preparation of compound 14 has been reported in reference 4, hereby incorporated by reference in its entirety for all purposes.

In some aspects, the compound of formula (I-c) is prepared as depicted in Scheme 9. Protecting groups and reaction conditions are the same as described elsewhere herein for the same or similarly structured compounds. The protecting groups and reaction conditions depicted in Scheme 9 are merely exemplary and not to be construed as limiting in any way. Preparation of compound 18 has been reported in reference 5, hereby incorporated by reference in its entirety for all purposes.

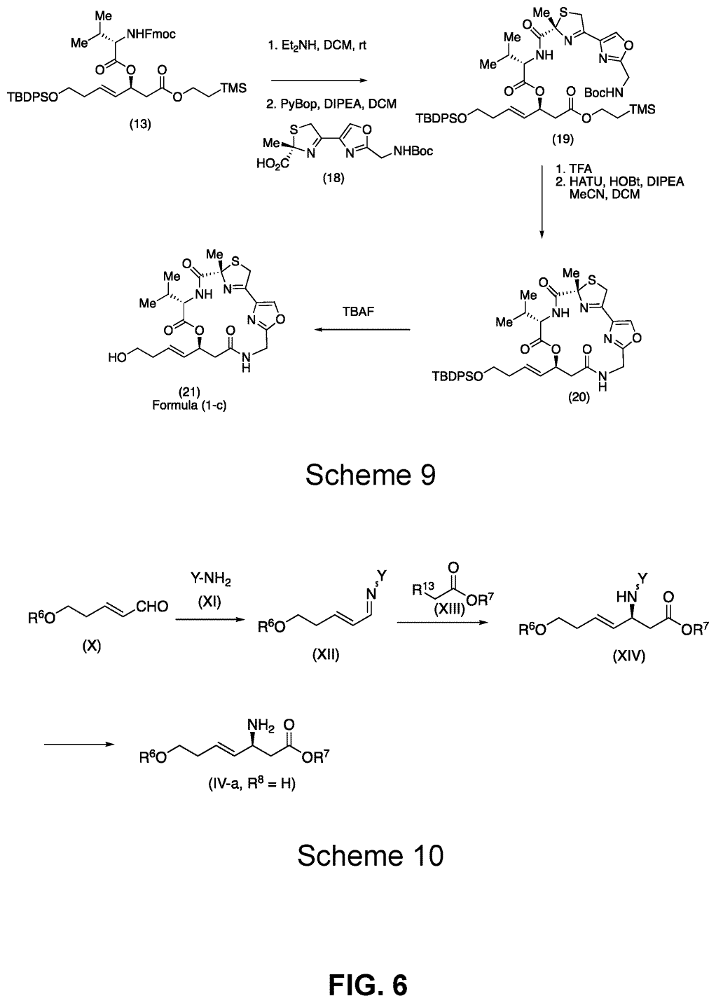

In some aspects, the compound of formula (IV-a, R⁸=H) is produced by a method, depicted in Scheme 10 of FIG. 6, comprising:
reacting via imine formation a compound of formula (X) with a chiral auxiliary of formula (XI) to form a compound of formula (XII);
reacting the compound of formula (XII) via nucleophilic addition with a compound of formula (XIII) to form the compound of formula (XIV);
   and cleaving N—Y bond to from the compound of formula (IV-a, R⁸=H), in which Y is a chiral group sufficient to effect a diastereoselective nucleophilic addition of the compound of formula (XIII) to a compound of formula (XII) to form the compound of formula (IV-a) as a majority product, in which R¹³ is Cl, Br, I, F, alkali metal, an alkali earth metal, ZnX, CuX, Cu, or a transition metal, in which each X in ZnX or CuX independently is a halide (e.g., F, Cl, Br, or I).

In some aspects, X is NH and the method further comprises producing the compound of formula (IV) by:
reacting via imine formation a compound of formula (X):

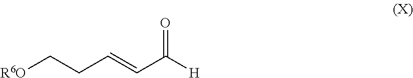

with a chiral auxiliary of formula (XI):

Y—NH₂ (XI)
to form a compound of formula (XII):

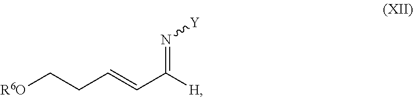

reacting the compound of formula (XII) via nucleophilic addition with a compound of formula (XIII):

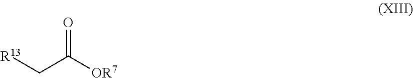

to form the compound of formula (IV) where X is NH and R⁸ is chiral group Y:

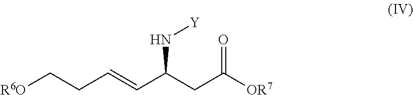

and cleaving the N—Y bond to form the compound of formula (IV) where R⁸ is H, and when X is N, the N is neutral or positively charged,
wherein:
Y is a chiral group sufficient to effect a diastereoselective nucleophilic addition of the compound of formula (XIII) to the compound formula (XII) to form the compound of formula (IV) as a majority product where R⁸ is chiral group Y,
R⁶ is a suitable alcohol protecting group,
R⁷ is a suitable carboxylic acid protecting group;
optionally wherein the carboxylic acid protecting group is selected from C₁-C₆ linear or branched alkyl, C₁-C₆ linear or branched alkyl substituted with one or more aryl or heteroaryl in any position, or alkylsilyl;
optionally wherein the C₁-C₆ linear or branched alkyl, or the C₁-C₆ linear or branched alkyl substituted with one or more aryl or heteroaryl in any position, is selected from methyl, ethyl, propyl, isopropyl, allyl, butyl, 2-butyl, tert-butyl, benzyl, or substituted benzyl; and
optionally wherein the alkylsilyl is 2-(trimethylsilyl)ethyl; and R¹³ is H, a halide, or a metal comprising Li, Na, K, ZnBr, ZnCl, CuCl, CuBr, CuI, Cu, or any combination thereof.

In some aspects, the chiral auxiliary of formula (XI) is:

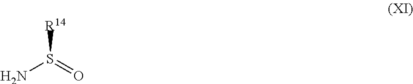

wherein R¹⁴ is C₁₋₆ linear and branched alkyl or aromatic or heteroaromatic. In some aspects, R¹⁰ is tert-butyl, p-tolyl, alkyl, an aromatic group, or any substituted versions thereof. In some aspects, the compound of formula (XI) is (R)-tert-butanesulfinamide.

In some aspects, the compound of formula (XIII) is bromoacetate ester, which is converted to the corresponding Reformatsky type intermediate in the presence of Zn metal with/without Cu(I) halides. The Reformatsky intermediate reacts with imine (XII) to form a compound of formula (XIV).

In some aspects, suitable solvents for Reformatsky type reaction are THF, 2-methyl THF, or a combination thereof.

In some aspects, suitable temperatures for Reformatsky include −20 to 50° C., −15 to 30° C., or 0 to 5° C.

In some aspects, cleavage of the N—Y bond can be done under acidic conditions, such as HCl. Suitable solvents for N—Y bond cleavage are 1,4-dioxane, diethyl ether, THF, MTBE, or a combination thereof.

In some aspects, suitable temperatures for N—Y bond cleavage include −10 to 50° C., −5 to 40° C., or 0 to 30° C.

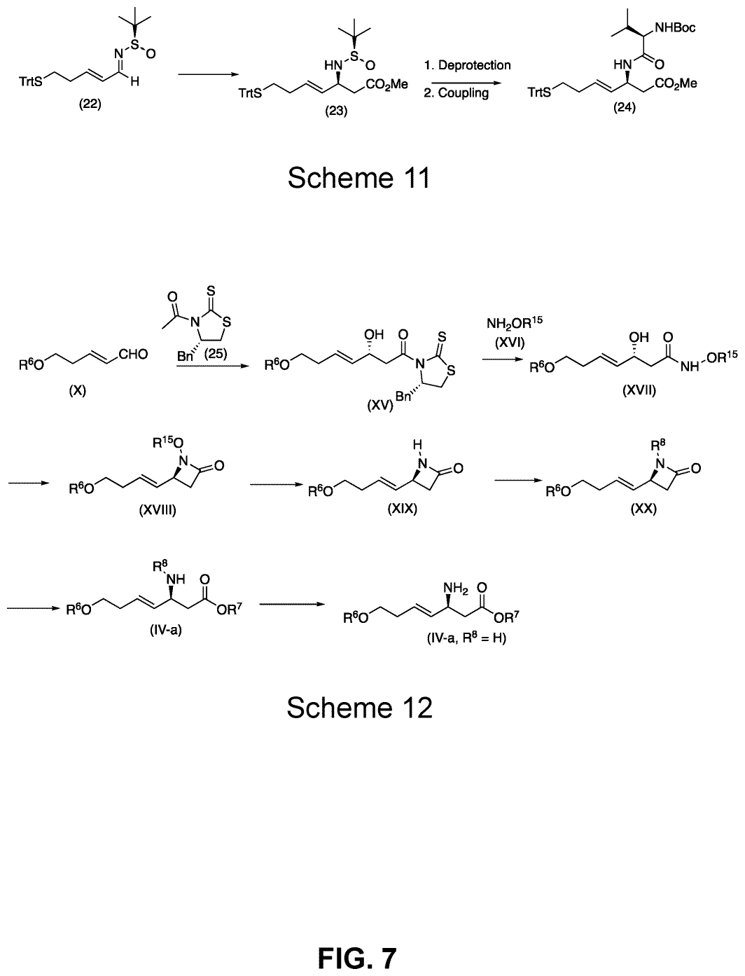

In some aspects, the synthetic method depicted in Scheme 10 in FIG. 6 is applied in the synthesis of the S-Trt protected compound 24 by replacing OR⁶ with S-Trt as depicted in Scheme 11 in FIG. 7. Preparation of compounds 1 from compound 24 have been reported in reference 1, and preparation of compound 22 has been reported in reference 6, both of which are hereby incorporated by reference in their entireties.

In some aspects, the compound of formula (IV-a) is produced by a method, depicted in Scheme 12 of FIG. 7, comprising:
reacting via Evans type diastereoselective aldol reaction between a compound of formula (X) with Evans chiral auxiliary 25 to form a compound of formula (XV) as a major product;
reacting a compound of formula (XV) with a compound of formula (XVI) to form a compound of formula (XVII);
reacting a compound of formula (XVII) under Mitsunobu reaction conditions to form a compound of formula (XVIII);
reducing N—O bond of a compound of formula (XVIII) to form a compound of formula (XIX);
protecting the resulting free nitrogen of a compound of formula (XIX) with a nitrogen protecting group to form a compound of formula (XX);
opening a beta-lactam of formula (XX) to form a compound of formula (IV-a),
removing the protecting group of a compound of formula (IV-a) to form a compound of formula (IV-a, R⁸═H);
wherein
R⁶, R⁷, and R⁸ are defined elsewhere herein; R¹⁵ is C₁-C₆ alkyl or benzyl. Formula (XVI) may be free base or its salts.

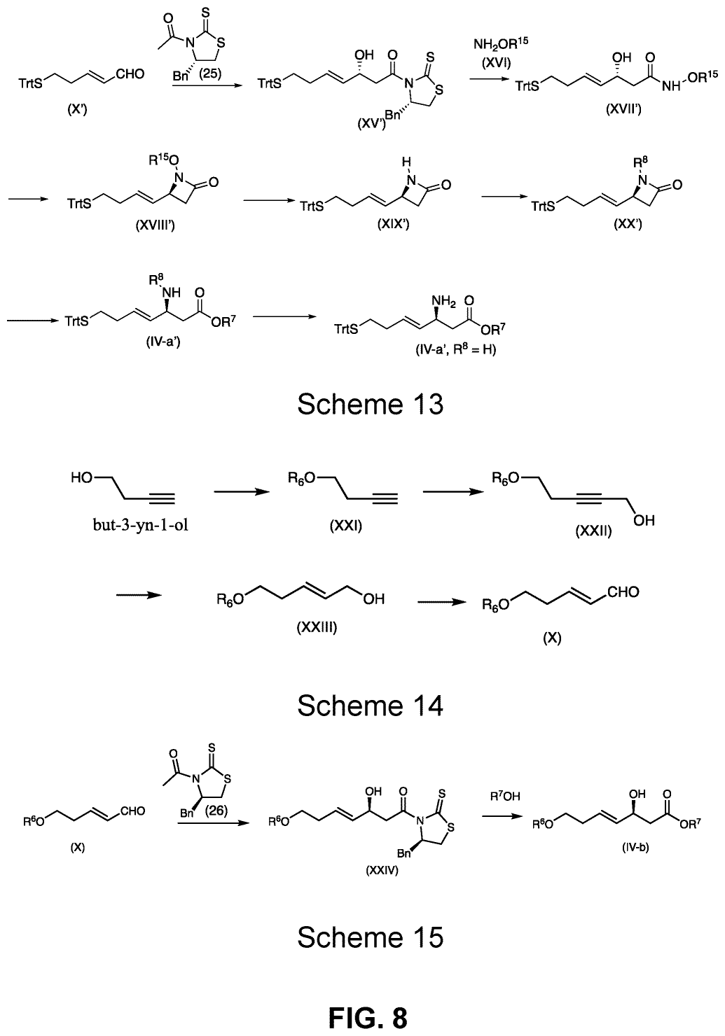

In some aspects, disclosed is a synthetic route depicted in Scheme 13 of FIG. 8 to prepare the corresponding S-Trt derivative (IV-a′, R⁸═H) from a compound (X′) by modifying the synthetic route in Scheme 12 of FIG. 7 by replacing OR⁶ with S-Trt. Preparation of compounds 1 from formula (IV-a′, R⁸═H) have been reported in reference 1,

In some aspects, the compound of formula (X) is produced as shown in Scheme 14 of FIG. 8 by a method comprising:
protecting hydroxyl group of but-3-yn-1-ol with a suitable protecting group to form a compound of formula (XXI),
reacting a compound of formula (XXI) with strong base, such as n-BuLi, LDA, LiHMDS, NaHMDS, KHMDS, EtMgX, MeMgX, iPrMgX, to generate the corresponding anion then trapped with formaldehyde, paraformaldehyde, or trioxane to form a compound of formula (XXII),
reducing a compound of formula (XXII) with DIBAL to form a compound of formula (XXIII), and
oxidizing a compound of formula (XXIII) to form a compound of formula (X).

In some aspects, the compound of formula (IV-b) is produced in Scheme 15 of FIG. 8 by a method comprising:
reacting via Evans type diastereoselective aldol reaction between a compound of formula (X) with Evans chiral auxiliary 26 to form a compound of formula (XXIV) as a major product; and
reacting a compound of formula (XXIV) with alcohol (R₇OH) to form a compound of formula (IV-b).

In some aspects, X is O, and the method comprises producing the compound of formula (IV) by:
reacting via an aldol addition a compound of formula (X):

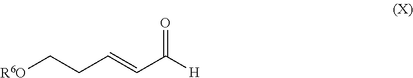

with a chiral auxiliary of formula (XXVIII):

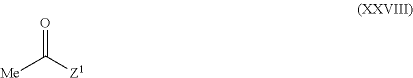

wherein Z¹ is a chiral group sufficient to effect a diastereoselective aldol addition to form a compound of formula (XXVIX):

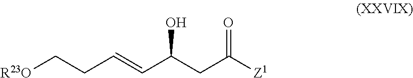

and performing an esterification on the compound of formula (XXVIX) to produce the compound of formula (IV) where X is O. In some aspects, the chirality of the Z¹ group controls the stereochemistry of a reaction by causing a particular diastereomer to be formed as the majority product in an aldol addition. While at least two products are possible in such an aldol addition, one diastereomeric product is formed in a greater yield than the other possible diastereomeric products. In some aspects, the compound of formula (XXVIII) is any known compound that can effect a such a diastereoselective aldol addition, such as an Evans-type oxazolidinone chiral auxiliary, Evans-type oxazolidinethione chiral auxiliary, Evans-type thiazolidinethione chiral auxiliary, or Crimmins-type thiazolidinethione chiral auxiliary.

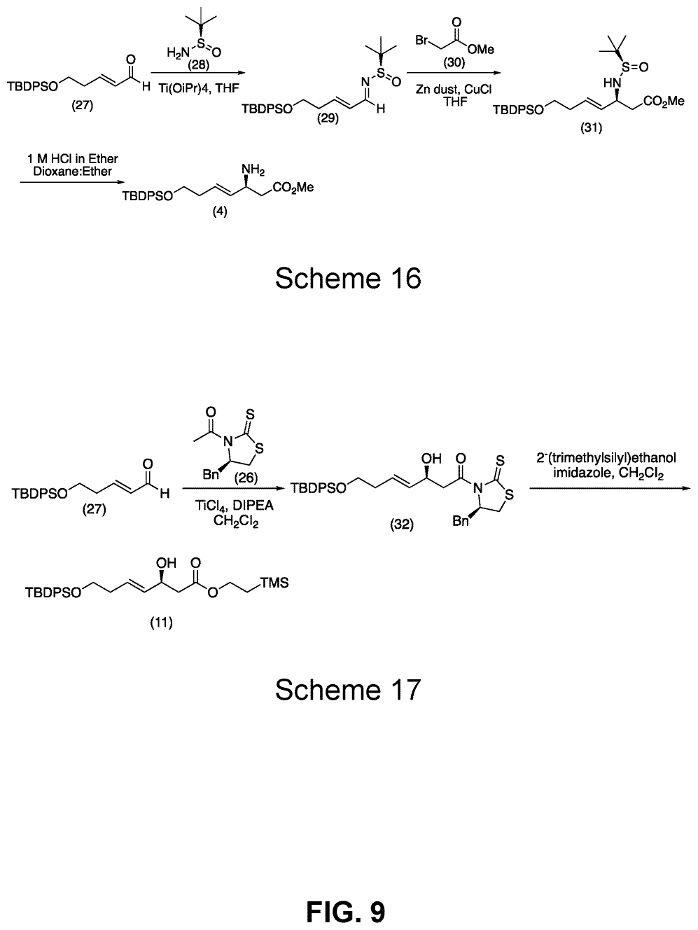

In some aspects, compound 4 is prepared as disclosed elsewhere herein for the same or similarly structured compounds, as shown in Scheme 16 of FIG. 9. The choice of reagents and protective groups is exemplary and not intended to be limiting in any way.

In some aspects, the synthetic method depicted in Scheme 10 of FIG. 6 is applied in the synthesis of TBDPS protected compound 4 by replacing R⁶ with TBDPS as depicted in Scheme 16 of FIG. 9. Preparation of compound 10 from compound 4 is described elsewhere herein for the same or similarly structured compounds.

In some aspects, compound 11 is prepared as described elsewhere herein for the same or similarly structured compounds, as shown in Scheme 17 of FIG. 9. The choice of reagents and protective groups is exemplary and not intended to be limiting in any way.

In some aspects, disclosed is a compound of formula (XXVI):

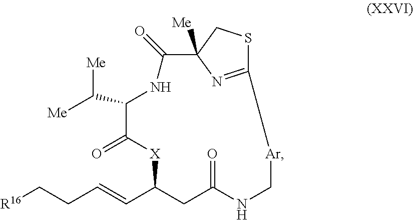

wherein:
X is O or NH;
Ar is thiazolyl, pyridinyl, or oxazolyl;
R¹⁶ is OH, OR₆, or L;
R⁶ is a suitable alcohol protecting group; and
L is a leaving group selected from C₁-C₂₄ linear or branched carbon sulfonate, substituted C₁-C₂₄ linear or branched sulfonate, arylsulfonate, substituted arylsulfonate, heteroarylsulfonate, substituted heteroarylsulfonate, phosphate, or halogen.

In some aspects, the compound of formula (XXVI) is a compound of formula (I-a), (I-b), or (I-c):

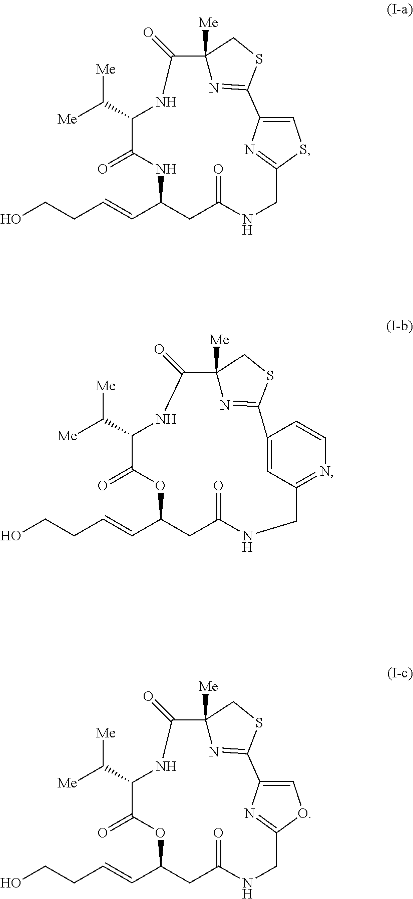 [SMILES: CC(C)[C@@H]1NC(=O)[C@]2(C)CSC(=N2)c2csc(n2)CNC(=O)C[C@@H](/C=C/CCO)NC1=O ; CC(C)[C@@H]1NC(=O)[C@]2(C)CSC(=N2)c2ccnc(c2)CNC(=O)C[C@@H](/C=C/CCO)OC1=O ; CC(C)[C@@H]1NC(=O)[C@]2(C)CSC(=N2)c2coc(n2)CNC(=O)C[C@@H](/C=C/CCO)OC1=O]

In some aspects, R¹⁶ is OH. In some aspects, R¹⁶ is L. In some aspects, R¹⁶ is OR₆.

In some aspects, X is O, and Ar is pyridinyl. In some aspects, X is NH, and Ar is pyridinyl. In some aspects, X is O, and Ar is oxazolyl. In some aspects, X is NH, and Ar is oxazolyl. In some aspects, X is NH, and Ar is thiazolyl. In some aspects, X is O, and Ar is thiazolyl.

In some aspects, disclosed is a compound of formula (XXVII):

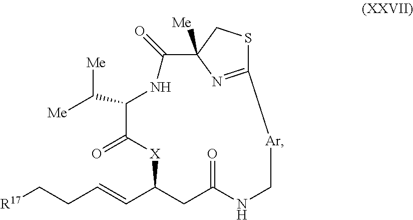

wherein:
X is O or NH;
Ar is thiazolyl, pyridinyl, or oxazolyl;
R₁₇ is R₂COS, R₃S, or R₄R₅N, and
R², R³, R⁴, and R⁵independently are hydrogen, C₁₋₂₄ linear alkyl (e.g., methyl, ethyl, n-propyl, n-butyl, n-pentyl, n-hexyl, n-heptyl, n-octyl and so forth), C₁-C₂₄ linear alkyl substituted with one or more aryl or heteroaryl in any position (e.g., substituted with phenyl, pyridinyl, furanyl, pyrrolyl, thiophenyl, and so forth, or any combination thereof), C₁-C₂₄ branched alkyl (e.g., isopropyl, isobutyl, sec-butyl, tert-butyl, and so forth), C₁-C₂₄ branched alkyl substituted with one or more aryl or heteroaryl in any position (e.g., isopropyl substituted with phenyl, isobutyl substituted with pyridinyl, and so forth), C₃-C₂₄ cyclic alkyl (e.g., cylopropyl, cyclobutyl, cyclopentyl, cyclohexyl, methylcyclopropyl, and so forth), C₃-C₂₄ cyclic alkyl substituted with one or more aryl or heteroaryl in any position (e.g., cyclohexyl substituted with phenyl, and so forth), C₁-C₂₄ alkenyl, C₁-C₂₄ alkynyl, aryl (e.g., naphthalenyl, phenyl, pryridinyl, furanyl, pyrrolyl, thiophenyl, and so forth), heteroaryl (e.g., pryridinyl, furanyl, pyrrolyl, thiophenyl, and so forth), diphenyl methyl, triphenylmethyl, or any substituted version thereof. In some aspects, the compound of formula (XXVII) excludes compounds where R² is unsubstituted C₇ linear alkyl when R₁₇ is R₂COS (i.e., a proviso where when R₁₇ is R₂COS then R² is not unsubstituted C₇ linear alkyl).

Various aspects are contemplated herein, several of which are set forth in the paragraphs below. It is explicitly contemplated that any aspect or portion thereof can be combined to form an aspect.

Aspect 1: A method for preparing a compound of formula (I):

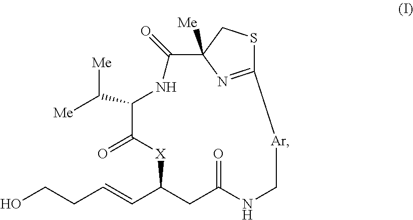

the method comprising reacting in one or more reactions:
a compound of formula (IV):

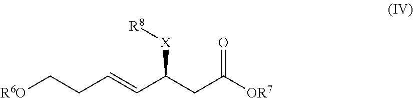

a compound of formula (V):

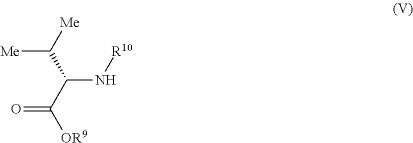

and a compound of formula (VI):

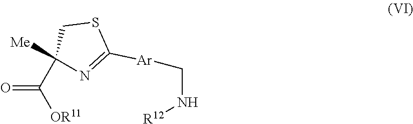

wherein:
X is O or NH;
Ar is thiazolyl, pyridinyl, or oxazolyl;
R⁶ is H or a suitable alcohol protecting group,
R⁸, R¹⁰, and R¹² independently are H, a suitable amine protecting group, or a chiral group,
R⁷, R⁹, and R¹¹ independently are H, an alkali metal ion, an alkaline earth metal ion, or a suitable carboxylic acid protecting group;
optionally wherein the suitable carboxylic acid protecting group is selected from C₁-C₆ linear or branched alkyl, C₁-C₆ linear or branched alkyl substituted with one or more aryl or heteroaryl in any position, or alkylsilyl;
optionally wherein the C₁-C₆ linear or branched alkyl, or the C₁-C₆ linear or branched alkyl substituted with one or more aryl or heteroaryl in any position, is selected from methyl, ethyl, propyl, isopropyl, allyl, butyl, 2-butyl, tert-butyl, benzyl, or substituted benzyl; and
optionally wherein the alkylsilyl is 2-(trimethylsilyl)ethyl.

Aspect 2: A method for preparing a compound of formula (III):

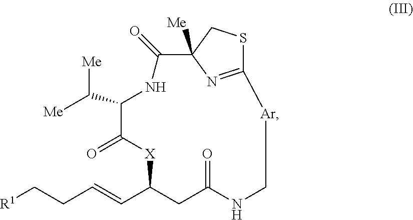

the method comprising:
reacting a compound of formula (I):

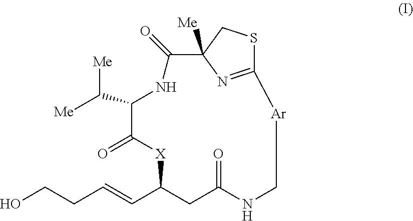

with an activating agent selected from C₁-C₂₀ linear or branched alkyl-sulfonyl halide, substituted alkyl-sulfonyl halide, arylsulfonyl halide, substituted arylsulfonyl halide, halo-phosphite (e.g., chlorophosphite), or a halogenating reagent, thereby forming a compound of formula (II):

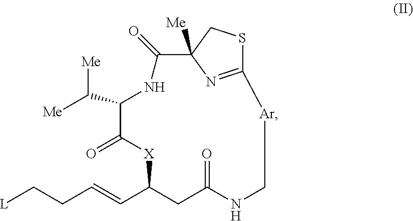

and reacting the compound of formula (II) with a nucleophile comprising an R¹ group, thereby forming the compound of formula (III);
wherein:
X is O or NH;
Ar is thiazolyl, pyridinyl, or oxazolyl;
R¹ is R₂COS, R₃S, or R₄R₅N;
R², R³, R⁴, and R⁵independently are hydrogen, C₁-C₂₄ linear alkyl, C₁-C₂₄ linear alkyl substituted with one or more aryl or heteroaryl in any position, C₁-C₂₄ branched alkyl, C₁-C₂₄ branched alkyl substituted with one or more aryl or heteroaryl in any position, C₃-C₂₄ cyclic alkyl, C₃-C₂₄ cyclic alkyl substituted with one or more aryl or heteroaryl in any position, C₁-C₂₄ alkenyl, C₁-C₂₄ alkynyl, aryl, heteroaryl, diphenyl methyl, triphenylmethyl, or any substituted version thereof; and
L is a leaving group selected from C₁-C₂₄ linear or branched carbon sulfonate, substituted C₁-C₂₄ linear or branched sulfonate, arylsulfonate, substituted arylsulfonate, heteroarylsulfonate, substituted heteroarylsulfonate, phosphate, or halogen.

Aspect 3: A method for preparing a compound of formula (III):

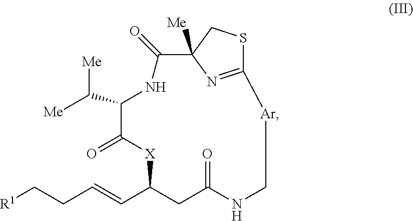

the method comprising:
reacting a compound of formula (II) with a nucleophile comprising an R¹ group:

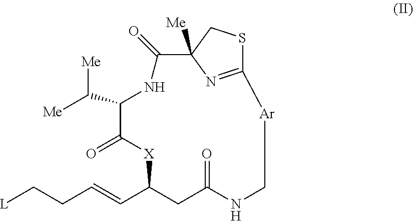

wherein:
X is O or NH;
Ar is thiazolyl, pyridinyl, or oxazolyl;
R¹ is R₂COS, R₃S, or R₄R₅N;
R², R³, R⁴, and R⁵independently are hydrogen, C₁-C₂₄ linear alkyl, C₁-C₂₄ linear alkyl substituted with one or more aryl or heteroaryl in any position, C₁-C₂₄ branched alkyl, C₁-C₂₄ branched alkyl substituted with one or more aryl or heteroaryl in any position, C₃-C₂₄ cyclic alkyl, C₃-C₂₄ cyclic alkyl substituted with one or more aryl or heteroaryl in any position, C₁-C₂₄ alkenyl, C₁-C₂₄ alkynyl, aryl, heteroaryl, diphenyl methyl, triphenylmethyl, or any substituted version thereof; and
L is a leaving group selected from C₁-C₂₄ linear or branched carbon sulfonate, substituted C₁-C₂₄ linear or branched sulfonate, arylsulfonate, substituted arylsulfonate, heteroarylsulfonate, substituted heteroarylsulfonate, phosphate, or halogen.

Aspect 4: The method of aspect 3, or any preceding aspect, further comprising producing the compound of formula (II) from a compound of formula (IX):

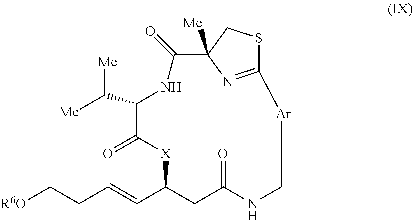

by converting OR⁶ of the compound of formula (IX) into the leaving group L;
wherein R⁶ is H or a suitable alcohol protecting group.

Aspect 5: The method of aspect 4, or any preceding aspect, wherein converting OR₆into the leaving group L comprises:
when R⁶ is the suitable alcohol protecting group, deprotecting the suitable alcohol protecting group to provide the compound of formula (IX) where R⁶ is H; and
reacting the compound of formula (IX) where R⁶ is H with an activating agent selected from C₁-C₂₀ linear or branched alkyl-sulfonyl halide, substituted alkyl-sulfonyl halide, arylsulfonyl halide, substituted arylsulfonyl halide, halo-phosphite (e.g., chlorophosphite), or a halogenating reagent.

Aspect 6: The method of aspect 4 or aspect 5, or any preceding aspect, wherein the compound of formula (IX) is a compound of formula (I):

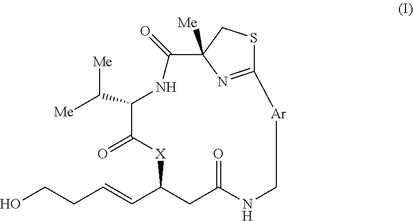

and the method comprises reacting the compound of formula (I) with an activating agent selected from C₁-C₂₀ linear or branched alkyl-sulfonyl halide, substituted alkyl-sulfonyl halide, arylsulfonyl halide, substituted arylsulfonyl halide, halo-phosphite (e.g., chlorophosphite), or a halogenating reagent, thereby forming the compound of formula (II).

Aspect 7: The method of any preceding aspect, wherein the compound of formula (I) or the compound of formula (IX) is a compound of formula (I-a), (I-b), or (I-c):

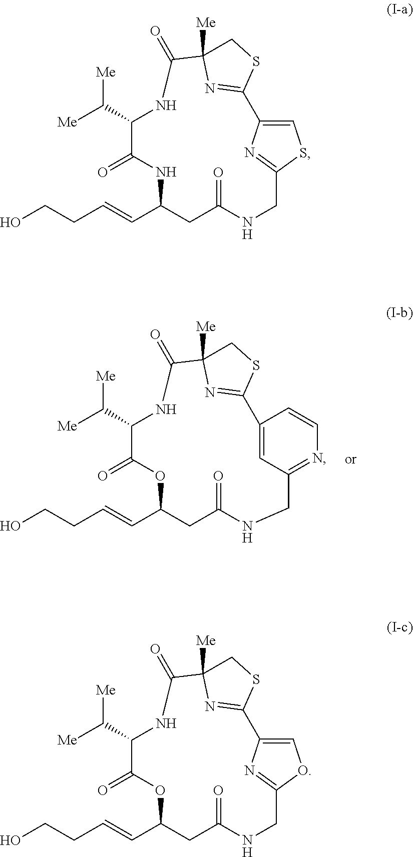 [SMILES: CC(C)[C@@H]1NC(=O)[C@]2(C)CSC(=N2)c2csc(n2)CNC(=O)C[C@@H](/C=C/CCO)NC1=O ; CC(C)[C@@H]1NC(=O)[C@]2(C)CSC(=N2)c2ccnc(c2)CNC(=O)C[C@@H](/C=C/CCO)OC1=O ; CC(C)[C@@H]1NC(=O)[C@]2(C)CSC(=N2)c2coc(n2)CNC(=O)C[C@@H](/C=C/CCO)OC1=O]

Aspect 8: The method of any preceding aspect, wherein L is OMs, OTs, OTf, 2,2,2-trifluoroethanesulfonate, n-octanesulfonate, benzenesulfonate, OP(═O)(OMe)₂, OP(═O)(OEt)₂, OP(═O)(OPh)₂, I, Br, Cl or F.

Aspect 9: The method of any preceding aspect, wherein Ar is:

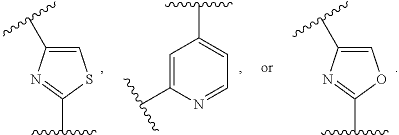 [SMILES: *c1csc(*)n1 ; *c1ccnc(*)c1 ; *c1coc(*)n1]

Aspect 10: The method of any preceding aspect, wherein the nucleophile is thiooctanoic acid, such that the compound of formula (III) is a compound of formula (XXV):

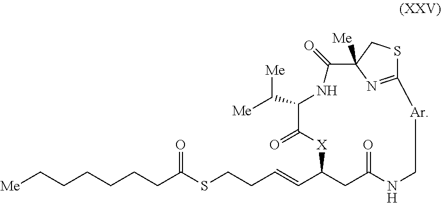

Aspect 11: The method of aspect 10, or any preceding aspect, wherein X is NH and Ar is:

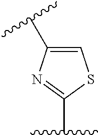 [SMILES: *c1csc(*)n1]

such that the compound of formula (III) is a compound of formula (1):

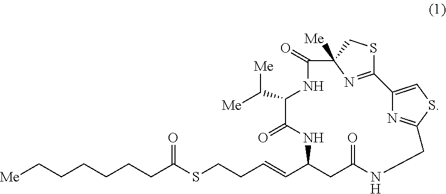 [SMILES: CCCCCCCC(=O)SCC/C=C/[C@@H]1CC(=O)NCc2nc(cs2)C2=N[C@@](C)(CS2)C(=O)N[C@@H](C(C)C)C(=O)N1]

Aspect 12: The method of aspect 10, or any preceding aspect, wherein X is O and Ar is:

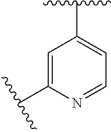 [SMILES: *c1ccnc(*)c1]

such that the compound of formula (III) is a compound of formula (2):

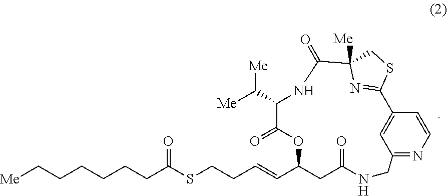 [SMILES: CCCCCCCC(=O)SCC/C=C/[C@@H]1CC(=O)NCc2cc(ccn2)C2=N[C@@](C)(CS2)C(=O)N[C@@H](C(C)C)C(=O)O1]

Aspect 13: The method of aspect 10, or any preceding aspect, wherein X is O and Ar is:

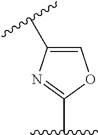 [SMILES: *c1coc(*)n1]

such that the compound of formula (III) is a compound of formula (3):

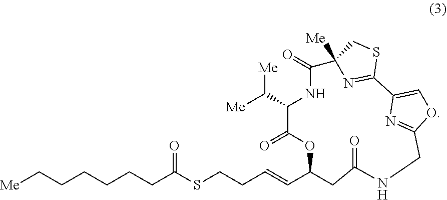 [SMILES: CCCCCCCC(=O)SCC/C=C/[C@@H]1CC(=O)NCc2nc(co2)C2=N[C@@](C)(CS2)C(=O)N[C@@H](C(C)C)C(=O)O1]

Aspect 14: The method of any one of aspects 2 or 4-13, or any preceding aspect, wherein the method further comprises producing the compound of formula (I) or the compound of formula (IX) from one or more reactions comprising:
a compound of formula (IV):

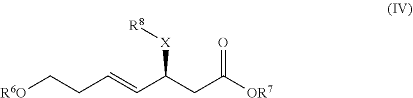

a compound of formula (V):

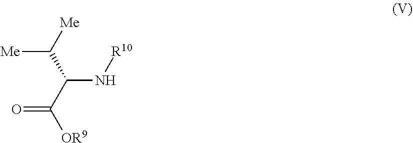

and a compound of formula (VI):

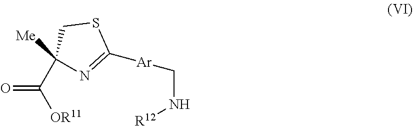

wherein:
R⁶ is H or a suitable alcohol protecting group,
R⁸, R¹⁰, and R¹² independently are H, a suitable amine protecting group, or a chiral group,
R⁷, R⁹, and R¹¹ independently are H, an alkali metal ion, an alkaline earth metal ion, or a suitable carboxylic acid protecting group;
optionally wherein the suitable carboxylic acid protecting group is selected from C₁-C₆ linear or branched alkyl, C₁-C₆ linear or branched alkyl substituted with one or more aryl or heteroaryl in any position, or alkylsilyl;
optionally wherein the C₁-C₆ linear or branched alkyl, or the C₁-C₆ linear or branched alkyl substituted with one or more aryl or heteroaryl in any position, is selected from methyl, ethyl, propyl, isopropyl, allyl, butyl, 2-butyl, tert-butyl, benzyl, or substituted benzyl; and
optionally wherein the alkylsilyl is 2-(trimethylsilyl)ethyl.

Aspect 15: The method of aspect 1 or aspect 14, or any preceding aspect, wherein:
the compound of formula (IV) is reacted with the compound of formula (V) via condensation to form a compound of formula (VII):

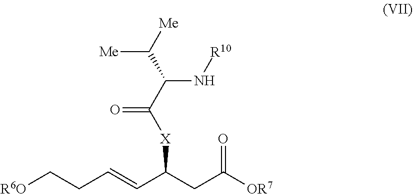

the compound of formula (VII) is reacted with the compound of formula (VI) via amidation to form a compound of formula (VIII):

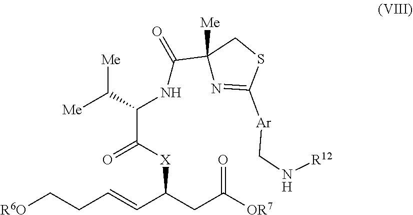

and the compound of formula (VIII) is reacted by amidating ring closure to form the compound of formula (IX) or the compound of formula (I) where R⁶ is H.

Aspect 16: The method of aspect 15, or any preceding aspect, wherein:
R⁶ is H or a suitable alcohol protecting group;
in reacting the compound of formula (IV) with the compound of formula (V): R⁸ is H, and when X is N, the N is neutral or positively charged; R⁷ is a suitable carboxylic acid protecting group; R¹⁰ is a suitable amine protecting group; and R⁹ is H, an alkali metal ion, or an alkaline earth metal ion;
in reacting the compound of formula (VI) with the compound of formula (VII): R⁷ is a suitable carboxylic acid protecting group; R¹⁰ is H, and optionally R¹⁰ is attached to a positively charged nitrogen atom; R¹¹ is H, an alkali metal ion, or an alkaline earth metal ion; and R¹² is a suitable amine protecting group; and
in reacting the compound of formula (VIII) by amidating ring closure: R⁸ is H, an alkali metal ion, or an alkaline earth metal ion, and when X is N, the N is neutral or positively charged; and R¹² is H, and optionally R¹² is attached to a positively charged nitrogen atom.

Aspect 17: The method of any one of aspects 1 or 14-16, or any preceding aspect, wherein X is NH, and the method further comprises producing the compound of formula (IV) by:
reacting via imine formation a compound of formula (X):

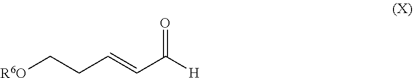

with a chiral auxiliary of formula (XI):

Y—NH₂ (XI)
to form a compound of formula (XII):

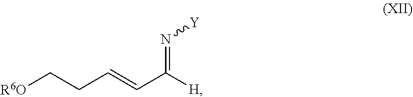

reacting the compound of formula (XII) via nucleophilic addition with a compound of formula (XIII):

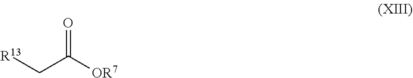

to form the compound of formula (IV) where X is NH and R⁸ is chiral group Y:

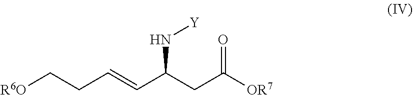

and cleaving the N—Y bond to form the compound of formula (IV) where R⁸ is H, and when X is N, the N is neutral or positively charged,
wherein:
Y is a chiral group sufficient to effect a diastereoselective nucleophilic addition of the compound of formula (XIII) to the compound formula (XII) to form the compound of formula (IV) as a majority product where R⁸ is chiral group Y,
R⁶ is a suitable alcohol protecting group,
R⁷ is a suitable carboxylic acid protecting group;
optionally wherein the carboxylic acid protecting group is selected from C₁-C₆ linear or branched alkyl, C₁-C₆ linear or branched alkyl substituted with one or more aryl or heteroaryl in any position, or alkylsilyl;
optionally wherein the C₁-C₆ linear or branched alkyl, or the C₁-C₆ linear or branched alkyl substituted with one or more aryl or heteroaryl in any position, is selected from methyl, ethyl, propyl, isopropyl, allyl, butyl, 2-butyl, tert-butyl, benzyl, or substituted benzyl; and
optionally wherein the alkylsilyl is 2-(trimethylsilyl)ethyl; and
R¹³ is H, a halide, or a metal comprising Li, Na, K, ZnBr, ZnCl, CuCl, CuBr, CuI, Cu, or any combination thereof.

Aspect 18: The method of aspect 17, or any preceding aspect, wherein the chiral auxiliary of formula (XI) is:

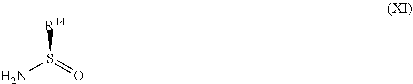

wherein R¹⁴ is tert-butyl, p-tolyl, alkyl, an aromatic group, or any substituted versions thereof.

Aspect 19: The method of aspect 18, or any preceding aspect, wherein R¹⁴ is tert-butyl, such that the compound of formula (XI) is (R)-tert-butanesulfinamide.

Aspect 20: A compound of formula (XXVI):

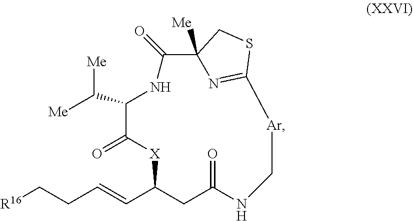

wherein:
X is O or NH;
Ar is thiazolyl, pyridinyl, or oxazolyl;
R¹⁶ is OH, OR₆, or L;
R⁶ is a suitable alcohol protecting group; and
L is a leaving group selected from C₁-C₂₄ linear or branched carbon sulfonate, substituted C₁-C₂₄ linear or branched sulfonate, arylsulfonate, substituted arylsulfonate, heteroarylsulfonate, substituted heteroarylsulfonate, phosphate, or halogen.

Aspect 21: The compound of aspect 20, or any preceding aspect, wherein the compound of formula (XXVI) is a compound of formula (I-a), (I-b), or (I-c):

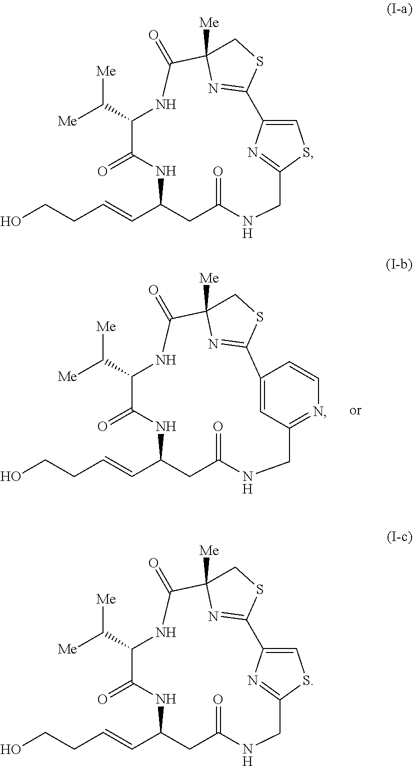 [SMILES: CC(C)[C@@H]1NC(=O)[C@]2(C)CSC(=N2)c2csc(n2)CNC(=O)C[C@@H](/C=C/CCO)NC1=O ; CC(C)[C@@H]1NC(=O)[C@]2(C)CSC(=N2)c2ccnc(c2)CNC(=O)C[C@@H](/C=C/CCO)NC1=O ; "or" ; CC(C)[C@@H]1NC(=O)[C@]2(C)CSC(=N2)c2csc(n2)CNC(=O)C[C@@H](/C=C/CCO)NC1=O]

Aspect 22: The compound of aspect 20, or any preceding aspect, wherein R¹⁶ is OH.

Aspect 23: The compound of aspect 20, or any preceding aspect, wherein R¹⁶ is L.

Aspect 24: The compound of aspect 20, or any preceding aspect, wherein R¹⁶ is OR₆.

Aspect 25: The compound of any one of aspects 20-24, or any preceding aspect, wherein: X is O; and Ar is pyridinyl.

Aspect 26: The compound of any one of aspects 20-24, or any preceding aspect, wherein: X is O; and Ar is oxazolyl.

Aspect 27: The compound of any one of aspects 20-24, or any preceding aspect, wherein: X is NH; and Ar is thiazolyl.

### EXAMPLES

General Methods. Unless otherwise noted, all materials were obtained from commercial sources and used without purification. All reactions requiring anhydrous conditions were performed under a positive pressure of argon using flame-dried glassware. Dichloromethane, tetrahydrofuran, methanol, triethylamine, and acetonitrile were degassed with argon and dried through a solvent purification system (J. C. Meyer of Glass Contour). Flash chromatography was performed on Merck silica gel Kieselgel 60 (230-400 mesh) from EM science with the indicated solvent. ¹H NMR and ¹³C NMR spectra were recorded on Varian 400 MHZ spectrometers unless indicated otherwise. Mass spectra were obtained on Agilent 6224 TOF. Optical rotations were recorded at 24° C. on an Autopol III Automatic Polarimeter (Rudolph Research, Fairfield, NJ). X-ray diffraction (XRD) was recorded on a Brucker D8 Discover DaVinci.

TABLE 1
HDAC Inhibition. Table 1 depicts in vitro histone deacetylase (HDAC) inhibition by thiol-macrocycle and hydroxy-macrocycle,
in which HDAC inhibition is shown as IC₅₀ (M).

| HDAC Class | Class 1 | | | |
|---|---|---|---|---|
| HDAC Isoform | 1 | 2 | 3 | 8 |
| 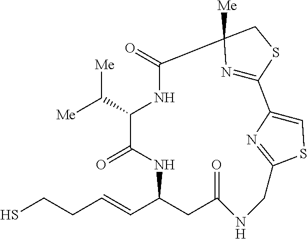 [SMILES: CC(C)[C@@H]1NC(=O)[C@]2(C)CSC(=N2)c2csc(n2)CNC(=O)C[C@@H](/C=C/CCS)NC1=O] | 4.42E−09 | 8.46E−09 | 2.49E−09 | 8.58E−07 |
| 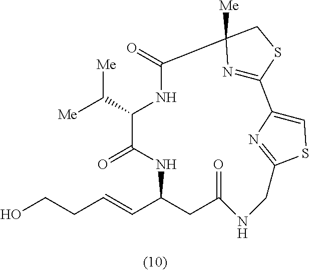 [SMILES: CC(C)[C@@H]1NC(=O)[C@]2(C)CSC(=N2)c2csc(n2)CNC(=O)C[C@@H](/C=C/CCO)NC1=O] | >1.00E−02 | >1.00E−02 | >1.00E−02 | >1.00E−02 |

| HDAC Class | Class 2a | | | | Class 2b | | Class 4 |
|---|---|---|---|---|---|---|---|
| HDAC Isoform | 4 | 5 | 7 | 9 | 6 | 10 | 11 |
| 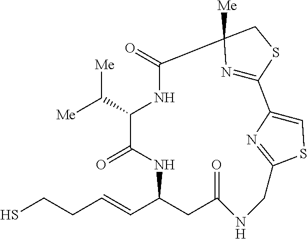 [SMILES: CC(C)[C@@H]1NC(=O)[C@]2(C)CSC(=N2)c2csc(n2)CNC(=O)C[C@@H](/C=C/CCS)NC1=O] | 1.00E−03¹ | 1.79E−04 | 1.00E−03¹ | 1.00E−03¹ | 1.38E−07 | n/a | 1.61E−04 |
| 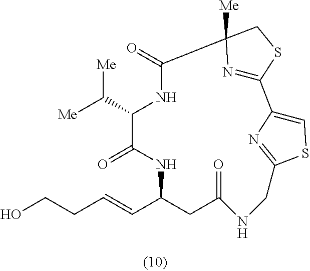 [SMILES: CC(C)[C@@H]1NC(=O)[C@]2(C)CSC(=N2)c2csc(n2)CNC(=O)C[C@@H](/C=C/CCO)NC1=O] | >1.00E−02 | >1.00E−02 | >1.00E−02 | >1.00E−02 | >1.00E−02 | n/a | >1.00E−02 |

HDAC Inhibition as IC₅₀ (M)
¹-Estimates of IC₅₀ values

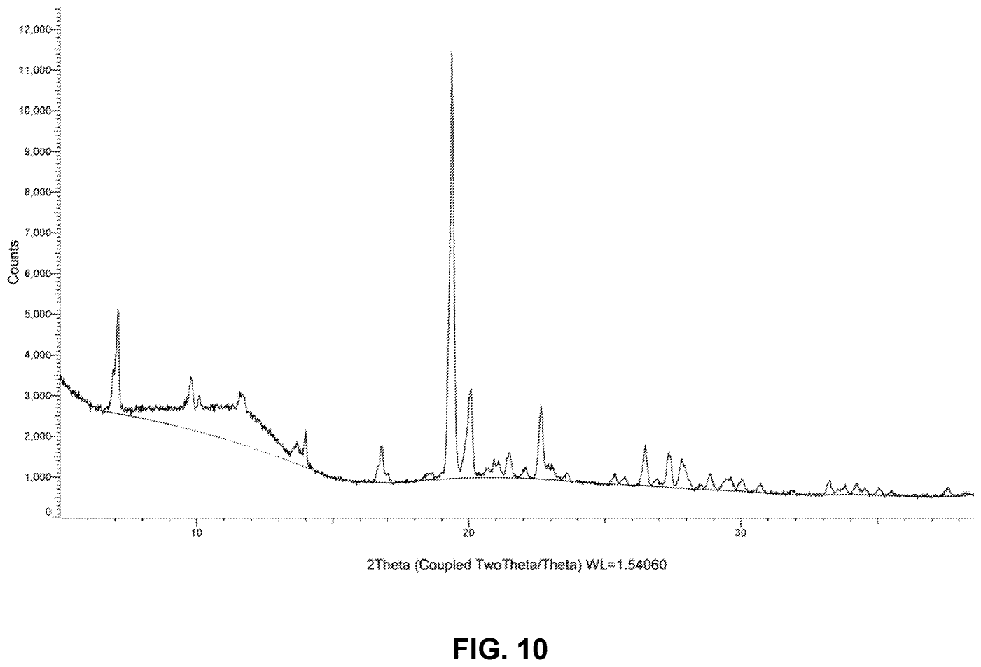

X-ray Diffraction. FIG. 10 is an XRD of compound 10 (i.e., compound of formula (I-a)).

Synthesis of Octanethioic S-acid. To the solution of thioacetamide (4.38 g, 58.3 mmol) in anhydrous toluene (75 mL) was added octanoyl chloride (10.0 mL, 58.3 mmol). The reaction mixture was stirred at 30° C. for 4 h. Then the reaction was cooled to 0° C. followed by addition of 10% aqueous NaOH (20 mL). The biphasic mixture was stirred at room temperature for 30 min and was acidified with aqueous 1M KHSO₄ to the pH 2-3. The acidified solution was extracted with ethyl acetate. The combined organic extracts were dried over anhydrous MgSO₄ and evaporated in vacuo. The crude product was filtered through silica to give thiooctanoic acid (7.30 g, 78% yield).

(E)-5-((tert-Butyldiphenylsilyl)oxy)pent-2-en-1-ol (34). To a neat mixture of 3-butyn-1-ol (10.0 g, 143 mmol) and imidazole (10.2 g, 171 mmol), tert-butyldiphenylchlorosilane (44.5 mL, 150 mmol) was added dropwise. The reaction mixture was stirred for 3.5 h. The precipitates were filtered and rinsed with diethyl ether. The combined organic extracts were dried over anhydrous Na₂SO₄. Evaporation of solvent in vacuo gave the crude (but-3-yn-1-yloxy)(tert-butyl)diphenylsilane, which was directly used without further purification.

To a solution of this crude product in anhydrous THF (600 mL) at −78° C., a solution of n-butyllithium (1.6 M, 98.1 mL, 157 mmol) in hexanes was added dropwise. The reaction mixture was stirred at −78° C for 1 h, warmed to room temperature over 2.5 h, and then cooled again to −78° C. To the reaction mixture, paraformaldehyde (5.57 g, 185 mmol) was added in one portion and the resulting suspension was slowly warmed to room temperature and stirred overnight. The mixture was concentrated in vacuo and then partitioned between diethyl ether and water. The phases were separated, and the aqueous phase was further extracted with diethyl ether. The combined organic extracts were dried over anhydrous Na₂SO₄ and concentrated in vacuo to give crude propargylic alcohol, which was directly used without further purification.

To a solution of crude propargylic alcohol in anhydrous diethyl ether (500 mL) at 0 ° C., Red-Al (70% in toluene) (45.6 mL, 152 mmol) was slowly added and the reaction mixture was stirred for 2 h at 0° C. The reaction mixture was quenched carefully with a wet paste of Na₂SO₄ until the appearance of colorless solid. The aluminum salts were filtered off, and the salts were washed repeatedly with hot THF. The combined filtrates were dried over anhydrous Na₂SO₄ and concentrated in vacuo. The residue was then purified by column chromatography on silica gel to give the trans-allylic alcohol 34 as a colorless oil (29.1 g, 60% yield over 3 steps). ¹H NMR (400 MHZ, CDCl₃) δ 7.73-7.64 (m, 4H), 7.48-7.35 (m, 6H), 5.74-5.62 (m, 2H), 4.07 (d, J=3.5 Hz, 2H), 3.73 (t, J=6.6 Hz, 2H),), 2.32 (q, J=6.1 Hz, 2H), 1.44 (bs, 1H), 1.07 (s, 9H); ¹³C NMR (101 MHZ, CDCl₃) δ 135.7, 134.1, 131.1, 129.7, 129.6, 127.7, 63.8, 63.6, 35.7, 27.0, 19.3.

(R)-N-((1E,2E)-5-((tert-Butyldiphenylsilyl)oxy)pent-2-en-1-ylidene)-2-methylpropane-2-sulfinamide (29). To a solution of DMSO (12.1 mL, 171 mmol) in anhydrous CH₂Cl₂ (200 mL) was added oxalyl chloride (10.8 mL, 128 mmol) over 15 min at −78° C. After stirring for 30 min at −78° C., trans-allylic alcohol 4 (29.1 g, 85.5 mmol) in anhydrous CH₂Cl₂ (500 mL) was added dropwise over 30 min and stirred at −78° C. for an additional 2.5 h. Then, triethylamine (35.7 mL, 256 mmol) was added dropwise and stirred at −78° C. for an additional hour and allowed to slowly warm to room temperature. After stirring at room temperature for 3 h, the reaction mixture was quenched by addition of water. The organic layer was separated and the aqueous layer was extracted with CH₂Cl₂. The combined organic extracts were dried over anhydrous Na₂SO₄ and concentrated in vacuo. The crude aldehyde (27) is immediately subjected to the next reaction without further purification

To a solution of this crude aldehyde in anhydrous THF (500 mL) was added Ti(OⁱPr)₄ (40.5 mL, 137 mmol) at room temperature and was stirred for 10 min. To this, (R)-tert-butanesulfinamide (11.4 g, 94.0 mmol) was added and the reaction stirred at room temperature overnight. The reaction was quenched with saturated aqueous NaCl. The crude mixture was filtered through Celite, and the filter cake was washed with ethyl acetate. The filtrate was then dried over Na₂SO₄, concentrated in vacuo, and the crude product was purified by column chromatography on silica gel (eluted by 15-85% ethyl acetate in hexanes) to give imine 29 (26.2 g, 71%). ¹H NMR (400 MHZ, CDCl₃) δ 8.18 (d, J=8.7 Hz, 1H), 7.65 (d, J=7.7 Hz, 4H), 7.41 (dq, J=14.0, 6.9 Hz, 6H), 6.60-6.42 (m, 2H), 3.80 (td, J=6.2, 2.1 Hz, 2H),), 2.51 (q, J=6.2 Hz, 2H), 1.20 (s, 9H), 1.05 (s, 9H); ¹³C NMR (101 MHZ, CDCl₃) δ 164,1, 148.3, 135.7, 133.7, 130.5, 129.9, 127.9, 62.4, 57.3, 36.4, 27.0, 22.6, 19.3; [α]²⁵_{D}: −161.6 (c=1.64, CHCl₃).

Methyl (S,E)-7-((tert-butyldiphenylsilyl)oxy)-3-(((R)-tert-butylsulfinyl)amino)hept-4-enoate (31). The oven dried flask was charged with Zn dust (34.0 g, 520 mmol) and CuCl (5.20 g, 52.0 mmol). The two solids were mixed under a slow stream of nitrogen while the flask was flame dried. The flask was allowed to cool to room temperature and anhydrous THF (120 mL) was added to produce a dark slurry. The resulting reaction mixture was heated to reflux and stirred vigorously for 30 min. The heating bath was then removed, and while maintaining vigorous stirring, methyl bromoacetate (30) (19.7 mL, 208 mmol) in anhydrous THF (40 mL) was added slowly via an addition funnel until reflux was re-initiated. The addition was continued at a rate that maintained a controllable reflux. The reaction mixture was stirred for an additional 30 min at room temperature after the addition was complete. Then the mixture was heated to 50° C. for 30 min. The reaction mixture was cooled to 0° C. To the reaction mixture was dropwisely added a solution of tert-butanesulfinyl imine 29 (23.0 g, 52.0 mmol) in anhydrous THF (120 mL) via an additional funnel at 0° C. The reaction mixture was stirred for an additional 18 h at 0° C. The reaction mixture was filtered through a pad of Celite, which was washed with diethyl ether. The filtrate was washed with saturated aqueous NaHCO₃, saturated aqueous NaCl. The organic layer was dried over Na₂SO₄ and concentrated in vacuo. The crude product was purified by column chromatography on silica gel (eluted by 50% ethyl acetate in hexanes) to give methyl ester 31 (20.1 g, 75%). ¹H NMR (400 MHZ, CDCl₃) δ 7.63-7.70 (m, 4H), 7.44-7.36 (m, 6H), 5.74 (dt, J=15.1, 6.9 Hz, 1H), 5.42 (ddt, J=15.4, 7.0, 1.4 Hz, 1H), 4.25 (d, J=5.7 Hz, 1H), 4.15 (p, J=6.2, 1H), 3.71-3.65 (m, 5H), 2.69 (dd, J=15.8, 4.9 Hz, 1H), 2.60 (dd, J=15.9, 7.1 Hz, 1H), 2.36-2.23 (m, 2H), 1.20 (s, 9H), 1.04 (s, 9H); ¹³C NMR (101 MHZ, CDCl₃) δ 172.0, 135.6, 135.6, 131.2, 130.4, 129.7, 127.7, 63.4, 55.6, 54.1, 51.9, 40.9, 36.0, 37.0, 22.8, 19.3; HRMS (ESI) m/z calcd for C₂₈H₄₁NNaO₄SSi [M+Na]⁺ 538.2423, found 538.2421; [α]²⁵_{D}: −45.8 (c=0.80, CHCl₃).

Methyl (S,E)-3-((S)-2-((tert-butoxycarbonyl)amino)-3-methylbutanamido)-7-((tert-butyldiphenylsilyl)oxy)hept-4-enoate (6). To a solution of 31 (5.5 g, 10.7 mmol) in anhydrous dioxane:ether (1:1, 100 mL), 1 N HCl in Et₂O (11 mL, 10.7 mmol) was added. The solution was filtered through a plug of basic alumina, and the solution was concentrated in vacuo. In a separate flask, PyBOP (8.32 g, 16.0 mmol) and DIPEA (5.6 mL, 32.0 mmol) were added to a solution of N-Boc-L-valine (5) (2.78 g, 12.8 mmol) in anhydrous CH₂Cl₂ (75 mL). The mixture was stirred for 5 min at room temperature before being cooled down to 0° C. The HCl salt form of the deprotected amine was dissolved in anhydrous CH₂Cl₂ (25 mL) and the solution was added dropwise to the activated acid at 0° C. under argon atmosphere. The mixture was warmed to room temperature and stirred for 8 h. The solvent was removed in vacuo and the crude reaction mixture was purified by column chromatography on silica gel (eluted by 30% ethyl acetate in hexanes) to yield di-peptide 6 (4.1 g, 63% yield over two steps). ¹H NMR (400 MHz, CDCl₃) δ 7.71-7.60 (m, 4H), 7.49-7.31 (m, 6H), 6.53 (d, J=8.4 Hz, 1H), 5.73-5.60 (m, 1H), 5.55-5.46 (m, 1H), 5.05 (d, J=5.4 Hz, 1H), 4.73-4.81 (m, 1H), 3.91-3.83 (m, 1H), 3.68-3.62 (m, 2H), 3.64 (s, 3H), 2.59 (d, J=5.4 Hz, 2H), 2.24-2.29 (q, J=6.7, 2H), 2.04-2.14 (m, 1H), 2.16-2.05 (m, 1H), 1.43 (s, 9H), 1.03 (s, 9H), 0.93 (d, J=6.8 Hz, 3H), 0.87 (d, J=6.8 Hz, 3H); ¹³C NMR (101 MHZ, CDCl₃) δ 171.9, 170.8, 135.7, 134.1, 133.9, 130.1, 129.8, 129.5, 127.8, 63.5, 51.9, 47.6, 38.9, 35.7, 31.2, 28.4, 27.0, 19.4, 19.3, 17.7; [α]²⁵_{D}: −5.3 (c=0.7, CHCl₃).

Methyl (S,E)-3-((S)-2-((R)-2′-(((tert-butoxycarbonyl)amino)methyl)-4-methyl-4,5-dihydro-[2,4′-bithiazole]-4-carboxamido)-3-methylbutanamido)-7-((tert-butyldiphenylsilyl)oxy)hept-4-enoate (8). Compound 6 (1.93 g, 3.20 mmol) was dissolved in anhydrous CH₂Cl₂ (50 mL) and treated with TFA (7 mL) at 0° C. The resulting solution was warmed to room temperature and stirred for 2 h. The solvent was evaporated in vacuo. The crude TFA salt was then dissolved in toluene, concentrated in vacuo, and dried on high vacuum to remove the residual TFA. In a separate flask, PyBOP (2.48 g, 4.76 mmol) and DIPEA (1.66 mL, 9.52 mmol) was added to a solution of 7 (1.36 g, 3.80 mmol) in anhydrous CH₂Cl₂ (30 mL). This stirred for 5 min at room temperature before being cooled down to 0° C. The crude TFA-salt was dissolved in anhydrous CH₂Cl₂ (15 mL) and was added dropwise to the solution of the activated acid. The reaction mixture was stirred at room temperature for 2 h. The mixture was dried in vacuo and the crude residue was immediately purified via column chromatography on silica gel (eluted by 40%-50% ethyl acetate in hexanes) to yield 8 (2.2 g, 2.58 mmol, 81% yield). ¹H NMR (400 MHZ, CDCl₃) δ 7.96 (s, 1H), 7.64 (d, J=8.8 Hz, 4H), 7.46-7.33 (m, 6H), 6.62 (d, J=8.7 Hz, 1H), 5.73-5.61 (m, 1H), 5.51 (dd, J=15.5, 6.3 Hz, 1H), 5.29 (s, 1H), 4.86-4.74 (m, 1H), 4.63 (d, J=6.3 Hz, 2H), 4.17 (dd, J=8.8, 6.3 Hz, 1H), 3.76 (d, J=11.6 Hz, 1H), 3.69-3.61 (m, 3H) 3.65 (s, 3H), 3.33 (d, J=11.6 Hz, 1H), 2.60 (d, J=5.3 Hz, 2H), 2.26 (q, J=6.6 Hz, 2H), 2.18-2.07 (m, 1H), 1.58 (s, 3H), 1.47 (s, 9H), 1.03 (s, 9H), 0.85 (dd, J=15.5, 6.8 Hz, 3H), 0.81 (d, J=6.8 Hz, 3H); ¹³C NMR (101 MHZ, CDCl₃) δ 174.8, 171.8, 169.9, 169.8, 163.2, 155.7, 135.6, 134.0, 133.9, 130.1, 129.7, 129.5, 127.7, 85.1, 80.5, 63.4, 58.6, 51.8, 47.6, 42.4, 41.6, 38.8, 35.7, 30.8, 28.4, 26.9, 24.8, 19.4, 19.3, 17.8; [α]²⁵_{D}: −39.5 (c=1.2, CHCl₃).

(12Z,22Z,24R,5S,8S)-8-((E)-4-((tert-butyldiphenylsilyl)oxy)but-1-en-1-yl)-5-isopropyl-24-methyl-24,25-dihydro-4,7,11-triaza-1(4,2),2(2,4)-dithiazolacyclododecaphane-3,6,10-trione (9). To a solution of methyl ester 8 (2.0 g, 2.40 mmol) in a THF:water (50 mL, 2:1) was added LiOH·H₂O (0.12 g, 4.6 mmol). The reaction was stirred for 30 min, The reaction mixture was cooled to 0° C. and acidified to PH ˜3-4 by dropwise addition of 1M aqueous KHSO₄. The solution was diluted with water and extracted with CH₂Cl₂. The combined organic extracts were dried over Na₂SO₄, and concentrated in vacuo to provide the corresponding acid, which was used without further purification. To a solution of the acid in anhydrous CH₂Cl₂ (100 mL) at 0° C. was added TFA (7 mL). The mixture was stirred at room temperature for 2 h. The solvent was removed in vacuo. The crude salt was then dissolved in toluene, concentrated in vacuo, and dried on high vacuum to remove residual TFA. To a solution of HATU (2.70 g, 7.10 mmol), HOBt·H₂O (0.650 g, 4.80 mmol) and DIPEA (2.4 mL, 14.2 mmol) in anhydrous CH₃CN (1850 mL) at 0° C. was added a solution the TFA salt in anhydrous CH₃CN (50 mL) dropwise over 20 h via a syringe pump at 0° C. After complete addition, the resulting mixture was stirred at 0° C. for 8 h and room temperature for an additional 15 h. The solvent of the reaction mixture was evaporated in vacuo. The resulting residue was dissolved in CH₂Cl₂ (50 mL), filtered through a Celite pad and dried over anhydrous MgSO₄. The solvent was evaporated in vacuo, and the resulting residue was purified by chromatography column on silica gel (eluted by 3% methanol in CH₂Cl₂) to give macrocyclic core 9 (1.08 g, with 64% yield). ¹H NMR (400 MHZ, CDCl₃) δ 7.78 (s, 1H), 7.64 (d, J=6.6 Hz, 4H), 7.43-7.34 (m, 6H), 6.50 (d, J=10.7 Hz, 1H), 6.38 (d, J=8.9 Hz 1H), 6.19 (d, J=8.2 Hz 1H), 5.75-5.63 (m, 1H), 5.50 (dd, J=15.5, 5.9 Hz, 1H), 5.24 (dd, J=17.6, 8.9 Hz, 1H), 4.98 (s, 1H), 4.53 (dd, J=10.7, 3.2 Hz, 1H), 4.24 (dd, J=17.7, 3.2 Hz, 1H), 3.90 (d, J=11.6 Hz, 1H), 3.70-3.62 (m, 2H), 3.37 (d, J=11.6 Hz, 1H), 2.72 (dd, J=15.4, 4.0 Hz, 1H) 2.55-2.20 (m, 2H), 2.27 (q, J=6.6 Hz, 2H), 1.86 (s, 3H), 1.04 (s, 9H), 0.77 (d, J=6.9 Hz, 3H), 0.36 (d, J=6.8 Hz, 3H);¹³C NMR (101 MHZ, CDCl₃) δ 173.8, 170.0, 169.9, 168.7, 165.9, 135.7, 134.0, 130.9, 129.8, 128.5, 127.8, 124.2, 84.7, 63.5, 58.3, 48.1, 43.9, 40.9, 40.8, 35.7, 32.1, 27.0, 24.3, 19.6, 19.4, 15.3; HRMS (ESI) m/z calcd for C₃₇H₄₇N₅NaO₄S₂Si [M+Na]⁺ 740.2736, found 740.2736; [α]²⁵_{D}: +23.4 (c=0.29, CHCl₃).

(12Z,22Z,24R,5S,8S)-8-((E)-4-hydroxybut-1-en-1-yl)-5-isopropyl-24-methyl-24,25-dihydro-4,7,11-triaza-1(4,2), 2(2,4)-dithiazolacyclododecaphane-3,6,10-trione (10). To a solution of compound 9 (1.00 g, 1.40 mmol) in CH₂Cl₂: methanol (1:1, 60 mL) at 0° C. was added p-toluenesulfonic acid monohydrate (148 mg). The reaction mixture was stirred for 12 h at room temperature. The reaction was quenched with solid NaHCO₃ (176 mg) and was filtered through a Celite pad, which was washed with CH₂Cl₂: methanol (1:1) followed by evaporation in vacuo. The crude residue was purified over column chromatography on silica gel (eluted by 5% methanol in CH₂Cl₂) to afford compound 10 as a colorless solid (0.600 g, 90% yield). ¹H NMR (400 MHZ, CDCl₃:CD₃OD=4:1) δ 7.76 (s, 1H), 7.40-7.34 (m, 1H), 6.51 (d, J=10.8 Hz, 1H), 5.59-5.50 (m, 1H), 5.40 (dd, J=15.5, 15.7 Hz, 1H), 5.22 (d, J=17.8 Hz, 1H), 4.79-4.74 (m, 1H), 4.50 (d, J=10.8 Hz, 1H), 4.22 (d, J=17.8 Hz, 1H), 3.87 (d, J=11.8 Hz, 1H), 3.56-3.49 (m, 2H), 3.30-3.41 (m, 1H), 2.92-2.96 (m, 1H), 2.57-2.48 (m, 2H), 2.40-2.32 (m, 1H), 2.20 (q, J=7.2, 6.8 Hz, 2H), 1.85 (s, 3H), 1.40-1.30 (m, 1H), 0.66 (d, J=6.9 Hz, 3H), 0.18 (d, J=6.8 Hz, 3H); ¹³C NMR (101 MHZ, CDCl₃: CD₃OD=4:1) δ 174.0, 170.8, 170.7, 168.5, 167.5, 146.6, 131.5, 128.2, 124.5, 84.0, 61.2, 58.3, 49.1, 43.6, 40.7, 40.5, 35.6, 32.3, 24.0, 19.4, 15.0; HRMS (ESI) m/z calcd. for C₂₁H₂₉N₅NaO₄S₂ [M+Na]⁺ 502.1559, found 502.1568; [α]²⁵_{D}: +79.7 (c=0.4, CH₃OH).

S-((E)-4-((12Z,22Z,24R,5S,8S)-5-isopropyl-24-methyl-3,6,10-trioxo-24,25-dihydro-4,7,11-triaza-1(4,2), 2(2,4)-dithiazolacyclododecaphane- 8-yl)but-3-en-1-yl)octanethioate (1). To a solution 10 (0.400 g, 0.800 mmol) in anhydrous CH₂Cl₂ (10 mL) at 0° C, triethylamine (0.240 mL, 1.60 mmol) was added, followed by dropwise addition of 1-octanesulfonyl chloride (0.240 mL, 1.00 mmol) over 5 min. The reaction mixture was stirred for further 15 min. Then, the reaction mixture was washed with ice-cold water, 0.01 N aqueous HCl, saturated aqueous Na₂CO₃ solution and saturated aqueous NaCl solution in turns. The organic layer was dried over Na₂SO₄ and evaporated to give as a colorless solid. The crude compound and anhydrous K₂CO₃ (0.240 g, 1.60 mmol) were added sequentially to the solution of thiooctanoic acid 10 (0.800 g, 2.40 mmol) in anhydrous acetone (2 mL). The reaction mixture was stirred at room temperature for 12 h, then was diluted with acetone (2 mL), filtered through Celite and rinse with 4 mL of acetone then concentrated in vacuo. The crude product was purified by column chromatography on silica gel to give 1 as a colorless solid (0.310 g, 64% yield) (eluted by 3% MeOH in CH₂Cl₂). ¹H NMR (400 MHZ, CDCl₃) δ 7.78 (s, 1H), 6.51 (d, J=10.9 Hz, 1H), 6.49-6.40 (m, 1H), 6.26 (d, J=8.2 Hz, 1H), 5.72-5.60 (m, 1H), 5.52 (dd, J=15.5, 5.7 Hz, 1H), 5.28 (d, J=17.6 Hz, 1H), 5.01-4.92 (m, 1H), 4.54 (dd, J=3.1, 10.8 Hz, 1H), 4.32 (dd, J=3.4, 17.7 Hz, 1H), 3.89 (d, J=11.8 Hz, 1H), 3.38 (d, J=11.6 Hz, 1H), 2.89 (t, J=7.1 Hz, 2H), 2.74 (dd, J=15.4, 3.9 Hz, 1H), 2.57-2.48 (m, 3H), 2.40-2.32 (m, 2H), 2.16 (q, J=7.2, 6.8 Hz, 1H), 1.89 (s, 3H), 1.71-1.56 (m, 2H), 1.38-1.20 (m, 8H), 0.91-0.80 (m, J=6.6 Hz, 3H), 0.77 (d, J=6.9 Hz, 3H), 0.35 (d, J=6.8 Hz, 3H); ¹³C NMR (101 MHZ, CDCl₃) δ 199.7, 173.8, 170.0, 169.7, 168.7, 147.4, 130.8, 129.5, 124.3, 84.9, 58.3, 47.9, 44.3, 43.9, 41.0, 40.8, 32.4, 32.1, 31.7, 29.0, 28.9, 28.3, 25.8, 24.3, 22.7, 19.6, 15.6, 14.2; HRMS (ESI) m/z calcd for C₂₉H₄₃N₅NaO₄S₃ 644.2375 [M+Na]⁺, found 644.2379; [α]²⁵_{D}: +21.2 (c=0.30, CHCl₃), (lit. [α]²⁵_{D}: +23.2 (c=0.25, CHCl₃)).

Compound 1 was also prepared from compound 10 using a similar method that employed toluenesulfonyl chloride instead of 1-octanesulfonyl chloride with 50% yield.

Compound 1 was also prepared from compound 10 under a similar method that employed methanesulfonyl chloride instead of 1-octanesulfonyl chloride with 61% yield.

(S,E)-1-((R)-4-Benzyl-2-thioxothiazolidin-3-yl)-7-((tert-butyldiphenylsilyl)oxy)-3-hydroxyhept-4-en-1-one (32). To a cooled (0° C.) solution of (R)-1-(4-benzyl-2-thioxothiazolidin-3-yl) ethenone (26) (7.90 g, 31.5 mmol) in dry CH₂Cl₂ (100 mL) was added titanium (IV) chloride (34.2 mmol, 3.60 mL) dropwise, and the solution was allowed to stir for 5 min, during which the reaction mixture turned yellow in color. DIPEA (5.90 mL, 34.2 mmol) was then added to the reaction mixture. The yellowish slurry turned dark red and was stirred for 20 min at 0° C. To this dark red solution, compound 27 (8.90 g, 26.3 mmol) in dry CH₂Cl₂ (10 mL) was added dropwise, and the reaction mixture was stirred for 1 h at 0° C. After completion of the reaction, the reaction was quenched with ammonium chloride solution and then extracted into CH₂Cl₂. The combined organic layer was dried with anhydrous Na₂SO₄ and concentrated in vacuo to yield a crude residue, which was purified by silica gel column chromatography (eluted by hexane/EtOAc, 8:2) to afford 32 (11.0 g, 71%). ¹H NMR (400 MHZ, CDCl₃) δ 7.73-7.64 (m, 4H), 7.48-7.29 (m, 11H), 5.85-5.74 (m, 1H), 5.68-5.58 (m, 1H), 5.40 (ddd, J=10.7, 7.0, 4.0 Hz, 1H), 4.73-4.59 (m, 1H), 3.74 (t, J=6.6 Hz, 2H), 3.62 (dd, J=17.7, 2.9 Hz, 1H), 3.46-3.18 (m, 4H), 3.12-2.98 (m, 1H), 2.91 (d, J=11.6 Hz, 1H), 2.40-2.28 (m, 2H), 1.08 (s, 9H). ¹³C NMR (101 MHZ, CDCl₃) δ 201.3, 172.4, 136.8, 136.1, 133.7, 132.8, 131.4, 129.5, 129.1, 128.6, 128.1, 127.7, 69.3, 67.0, 63.3, 45.8, 37.4, 36.2, 34.9, 31.6, 26.8. HRMS (ESI) m/z calcd for C₃₃H₃₉NNaO₃S₂Si 612.2038 [M+Na]⁺, found 612.2024.

2-(Trimethylsilyl)ethyl (S,E)-7-((tert-butyldiphenylsilyl)oxy)-3-hydroxyhept-4-enoate (11). To a solution of thiazoline-thione 32 (9.30 g, 15.8 mmol) was dissolved in 150 mL CH₂Cl₂ was added 2-trimethylsilylethanol (22.6 mL, 158 mmol), followed by imidazole (1.60 g, 23.6 mmol). The resulting solution was stirred overnight. The reaction mixture was concentrated in vacuo and purified by flash-column chromatography (eluted by hexanes/EtOAc, 8:1) to provide compound 11 (7.10 g, 91%) as a clear oil. ¹H NMR (400 MHZ, CDCl₃) δ 7.51-7.30 (m, 10H), 5.66-5.58 (m, 1H) 5.43 (dd, J=15.3, 7.5 Hz 1H), 4.65-4.51 (m, 1H), 4.22-4.10 (m, 3H), 3.64 (t, J=6.6 Hz, 2H), 2.61 (dd, J=16.8, 6.1 Hz, 1H), 2.47 (dd, J=16.8, 8.0 Hz, 1H) 2.24-2.36 (m, 2H), 1.06 (s, 9H), 0.97-0.92 (m, 2H), 0.03 (s, 9H). ¹³C NMR (101 MHZ, CDCl₃) δ 171.9, 135.7, 133.8, 132.3, 131.8, 129.3, 127.4, 69.3, 67.0, 63.3, 46.4, 33.8, 31.5, 26.8, 19.4, −1.3.

2-(Trimethylsilyl)ethyl (S,E)-3-(((((9H-fluoren-9-yl)methoxy)carbonyl)-L-valyl)oxy)-7-((tert-butyldiphenylsilyl)oxy)hept-4-enoate (13). To a solution of compound 11 (7.00 g, 14.0 mmol) in CH₂Cl₂ (150 mL) at room temperature was added N-Fmoc-L-valine (12) (14.2 g, 42.1 mmol), EDCl-HCl (10.8 g, 56.1 mmol), DMAP (0.17 g, 1.40 mmol), and DIPEA (9.80 mL, 56.1 mmol). After stirring for 18 h, the reaction mixture was concentrated in vacuo. The crude residue was purified by flash-column chromatography (eluted by hexanes/EtOAc, 9:1) to provide compound 13 (7.34 g, 75%) as a clear oil. ¹H NMR (400 MHZ, CDCl₃)δ 7.74 (d, J=7.6 Hz, 2H), 7.68-7.51 (m, 6H), 7.45-7.26 (m, 10H), 5.83 (dt, J=14.5, 6.9 Hz, 1H), 5.66 (td, J=7.7, 5.4 Hz, 1H), 5.49 (dd, J=15.5, 7.5 Hz, 1H), 5.28 (d, J=9.1 Hz, 1H), 4.49-4.05 (m, 4H), 3.65 (t, J=6.6 Hz, 2H), 2.68 (dd, J=15.8, 8.0 Hz, 1H), 2.54 (dd, J=15.7, 5.5 Hz, 1H), 2.08-2.31 (m, 3H), 1.02 (s, 9H), 0.98-0.84 (m, 5H), 0.80 (d, J=6.9 Hz, 3H), −0.01 (s, 9H). ¹³C NMR (101 MHZ, CDCl₃) δ 170.8, 169.6, 156.1, 143.9, 143.8, 141.2, 135.5, 133.7, 132.6, 129.6, 128.2, 127.6, 127.6, 127.0, 125.1, 119.9, 72.0, 66.9, 63.0, 58.7, 47.2, 39.7, 35.5, 31.3, 26.8, 19.1, 18.9, 17.2, 14.1, −1.6.

2-(Trimethylsilyl)ethyl (S,E)-3-((((R)-2-(6-(((tert-butoxycarbonyl)amino) methyl)pyridin-2-yl)-4-methyl-4,5-dihydrothiazole-4-carbonyl)-L-valyl)oxy)-7-((tert-butyldiphenylsilyl)oxy)hept-4-enoate (15). Compound 13 (1.10 g, 1.57 mmol) was dissolved in anhydrous CH₃CN (50 mL) and treated with Et₂NH (5 mL) at room temperature under argon. The resulting solution was warmed to room temperature and stirred for 2 h. The solvent was evaporated in vacuo. The resulted crude compound was then dissolved in EtOAc, concentrated in vacuo, and dried on high vacuum to remove the residual Et₂NH. In a separate flask, PyBOP (1.23 g, 2.36 mmol) and DIPEA (0.55 mL, 3.15 mmol) was added to a solution of pyridine thiazolene fragment² 14 (0.66 g, 1.89 mmol) in anhydrous CH₂Cl₂ (30 mL) under argon at room temperature. This stirred for 5 min at room temperature before being cooled down to 0° C. The amine was dissolved in anhydrous CH₂Cl₂ (15 mL) and was added dropwise to the solution of the activated acid at 0° C. under argon. The reaction mixture was stirred at room temperature for 4 h. The mixture was concentrated in vacuo and the crude residue was immediately purified via column chromatography on silica gel (eluted by hexanes/EtOAc, 7:3) to yield 15 (1.04 g, 71%). ¹H NMR (400 MHZ, CDCl₃) δ 8.64-8.57 (m, 1H), 7.68-7.53 (m, 6H), 7.43-7.30 (m, 6H), 7.13 (d, J=9.0 Hz, 1H), 5.83 (dt, J=14.7, 6.8 Hz, 1H), 5.71-5.60 (m, 1H), 5.57-5.44 (m, 2H), 4.54-4.40 (m, 3H), 4.21-4.06 (m, 2H), 3.81 (d, J=11.6 Hz, 1H), 3.64 (t, J=6.7 Hz, 2H), 3.37 (dd, J=11.5, 1.9 Hz, 1H), 2.75-2.61 (m, 1H), 2.55 (dd, J=15.8, 5.5 Hz, 1H), 2.31-2.06 (m, 3H), 1.55 (s, 3H), 1.44 (s, 9H), 1.06-0.87 (m, 11H), 0.81 (d, J=7.1 Hz, 3H), 0.73 (d, J=7.1 Hz, 3H), −0.01 (d, J=1.9 Hz, 9H). ¹³C NMR (101 MHZ, CDCl₃) δ 173.9, 170.3, 169.7, 167.0, 149.8, 140.4, 135.5, 133.7, 132.6, 129.6, 128.2, 127.6, 120.3, 119.9, 85.4, 72.0, 63.1, 63.1, 56.8, 41.7, 39.8, 35.5, 31.1, 28.4, 26.8, 24.5, 19.1, 19.0, 17.3, 17.3, −1.5. HRMS (ESI) m/z calcd. for C₄₉H₇₁N₄O₈SSi₂ [M+H]⁺ 931.4531, found 931.4540.

(14R,7S, 10S,Z)-7-((E)-4-((Tert-butyldiphenylsilyl)oxy)but-1-en-1-yl)-10-isopropyl-14-methyl-14, 15-dihydro-8-oxa-4,11-diaza-1(2,4)-thiazola- 2(2,6)-pyridinacyclododecaphane-5,9,12-trione (16). To a solution of the compound 15 (0.10 g, 0.11 mmol) in anhydrous CH₂Cl₂ (10 mL) at 0° C. was added TFA (1 mL) under argon. The mixture was stirred at room temperature for 12 h. The solvent was removed in vacuo. The resulted crude salt was then dissolved in toluene, concentrated in vacuo, and dried on high vacuum to remove residual TFA. To a solution of HATU (0.12 g, 0.32 mmol), HOBt·H₂O (0.03 g, 0.21 mmol) and DIPEA (0.10 mL, 0.54 mmol) in anhydrous CH₃CN/CH₂Cl₂ (120 mL, 1:1) at room temperature was added a solution the TFA salt in anhydrous CH₃CN/CH₂Cl₂ (10 mL, 1:1) dropwise over 10 h via a syringe pump at 0° C. After complete addition, the resulting mixture was stirred for 8 h. The solvent of the reaction mixture was evaporated in vacuo. The resulting residue was dissolved in CH₂Cl₂ (5 mL), filtered through a Celite pad and dried over anhydrous MgSO₄. The solvent was evaporated in vacuo, and the resulting residue was purified by column chromatography on silica gel (eluted by 3% CH₃OH in CH₂Cl₂) and the solution was evaporated to dryness in vacuo and to give macrocyclic core 16 (15.0 mg, 20% yield). ¹H NMR (400 MHZ, CDCl₃) δ 8.69 (d, J=5.1 Hz, 1H), 7.80 (s, 1H), 7.64 (dq, J=6.4, 1.7 Hz, 4H), 7.44-7.35 (m, 6H), 6.91 (d, J=9.1 Hz, 1H), 6.11 (dd, J=8.4, 4.7 Hz, 1H), 5.93 (dtd, J=15.1, 6.9, 1.0 Hz, 1H), 5.80-5.68 (m, 1H), 5.66-5.58 (m, 1H), 5.09 (dd, J=17.6, 8.4 Hz, 1H), 4.58 (dd, J=9.1, 3.6 Hz, 1H), 4.22 (dd, J=17.6, 4.6 Hz, 1H), 4.06 (d, J=11.5 Hz, 1H), 3.76-3.66 (m, 2H), 3.38 (d, J=11.6 Hz, 1H), 3.22-3.15 (m, 2H), 2.81-2.72 (m, 1H), 2.39-2.10 (m, 3H), 1.78 (s, 3H), 1.04 (s, 9H), 0.75 (d, J=6.9 Hz, 3H), 0.65 (d, J=6.8 Hz, 3H). ¹³C NMR (101 MHZ, CDCl₃+CD₃OD) δ 173.9, 170.5, 170.1, 168.4, 167.3, 146.5, 135.5, 135.4, 133.8, 131.2, 129.6, 127.7, 127.6, 124.2, 84.0, 63.4, 58.1, 48.7, 43.6, 35.5, 32.2, 26.8, 24.1, 19.4, 19.1, 14.9. HRMS (ESI) m/z calcd. for C₃₉H₄₈N₄NaO₅SSi [M+Na]⁺ 735.3012, found 735.3021.

(14R,7S,10S,Z)-7-((E)-4-Hydroxybut-1-en-1-yl)-10-isopropyl-14-methyl-14,15-dihydro-8-oxa-4,11-diaza-1(2,4)-thiazola-2(2,6)- pyridinacyclododecaphane-5,9,12-trione (17). To a solution of compound 16 (15.0 mg, 0.02 mmol) in THF (2 mL) at 0° C. was added 1 M TBAF solution (0.7 ml, 0.07 mmol) under argon. The reaction mixture was stirred for 4 h at room temperature. The reaction was quenched with aq. NH₄Cl and was work up with CH₂Cl₂ (3×2 mL) followed by evaporation in vacuo. The crude residue was purified over column chromatography on silica gel (eluted by 5% CH₃OH in CH₂Cl₂) to afford compound 17 as a viscous liquid (9.0 mg, 90% yield). ¹H NMR (400 MHZ, CDCl₃) δ 8.63 (d, J=5.0 Hz, 1H), 8.42 (t, J=6.0 Hz, 1H), 7.88 (s, 1H), 7.35-7.29 (m, 1H), 7.12 (d, J=9.0 Hz, 1H), 6.00-5.72 (m, 3H), 4.86 (dd, J=17.3, 6.6 Hz, 1H), 4.53 (dd, J=9.0, 4.4 Hz, 1H), 4.41 (dd, J=17.3, 5.5 Hz, 1H), 35 3.98 (d, J=11.5 Hz, 1H), 3.75-3.50 (m, 2H), 3.06 (dd, J=15.6, 6.9 Hz, 1H), 2.83 (dd, J=15.6, 4.1 Hz, 1H), 2.49-2.04 (m, 4H), 1.78 (s, 3H), 0.81 (d, J=6.9 Hz, 3H), 0.73 (d, J=6.8 Hz, 3H). ¹³C NMR (101 MHZ, CDCl₃+CD₃OD) δ 173.5, 170.6, 169.3, 168.6, 159.6, 150.1, 140.1, 131.1, 129.0, 120.9, 117.3, 84.3, 72.5, 60.8, 58.0, 43.9, 43.3, 40.0, 35.3, 33.4, 24.6, 18.8, 17.0. HRMS (ESI) m/z calcd. For C₂₃H₃₀N₄NaO₅S [M+Na]⁺ 497.1835, found 497.1828.

S-((E)-4-((14R,7S,10S,Z)-10-Isopropyl-14-methyl-5,9,12-trioxo-14,15-dihydro-8-oxa-4,11-diaza-1(2,4)-thiazola-2(2,6)-pyridinacyclododecaphane- 7-yl)but-3-en-1-yl) octanethioate (2). (CAS Number: 1800584-53-5)4: Compound 2 was obtained using the same procedure for the preparation of compound 1, except that 17 was used instead of 10, (7.5 mg, 64% yield). ¹H NMR (400 MHZ, CDCl₃) δ 8.70 (d, J=5.0 Hz, 1H), 7.81 (s, 1H), 7.39 (dd, J=5.0, 1.6 Hz, 1H), 6.92 (d, J=9.1 Hz, 1H), 6.21 (dd, J=8.4, 4.8 Hz, 1H), 5.89 (dt, J=15.4, 6.8 Hz, 1H), 5.81-5.69 (m, 1H), 5.64 (dd, J=15.5, 6.7 Hz, 1H), 5.12 (dd, J=17.6, 8.4 Hz, 1H), 4.60 (dd, J=9.1, 3.7 Hz, 1H), 4.29 (dd, J=17.6, 4.7 Hz, 1H), 4.06 (d, J=11.6 Hz, 1H), 3.39 (d, J=11.6 Hz, 1H), 3.07-2.73 (m, 4H), 2.52 (t, J=7.6 Hz, 2H), 2.41-2.26 (m, 2H), 2.26-2.07 (m, 1H), 1.82 (s, 3H), 1.72-1.54 (m, 2H), 1.40-1.10 (m, 8H), 0.97-0.84 (m, 3H), 0.76 (d, J=6.9 Hz, 3H), 0.66 (d, J=6.8 Hz, 3H). ¹³C NMR (101 MHZ, CDCl₃) δ 199.3, 173.1, 170.1, 168.5, 168.3, 159.3, 150.8, 140.1, 132.7, 128.4, 120.9, 116.9, 84.6, 72.0, 57.9, 44.4, 44.1, 43.6, 40.5, 33.7, 32.3, 31.6, 28.9, 27.9, 25.6, 24.9, 22.6, 18.9, 17.1, 14.0. HRMS (ESI) m/z calcd for C₃₁H₄₅N₄O₅S₂ [M+H]⁺ 617.2826 found 617.2829; [α]²⁵_{D}: +86.9 (c=0.6, CHCl₃), (lit. [α]²⁵_{D}: +94.3 (c=0.62, CHCl₃)).

2-(Trimethylsilyl)ethyl (S,E)-3-((((R)-2-(2-(((tert-butoxycarbonyl)amino) methyl)oxazol-4-yl)-4-methyl-4,5-dihydrothiazole-4-carbonyl)-L-valyl)oxy)-7-((tert- butyldiphenylsilyl)oxy)hept-4-enoate (19). Compound 19 was obtained using the same procedure for the preparation of compound 15, except that 18 was used instead of 14 and with elution by 50% ethyl acetate in hexanes, (0.79 g, 71% yield). The yield is in relation to base fragment (compound 13). ¹H NMR (400 MHZ, CDCl₃) δ 8.12 (s, 1H), 7.68-7.60 (m, 4H), 7.32-7.44 (m, 6H), 7.11 (d, J=9.0 Hz, 1H), 5.84 (dt, J=14.4, 6.9 Hz, 1H), 5.61-5.69 (m, 1H), 5.56-5.45 (m, 1H), 5.19 (bs, 1H), 4.43-4.52 (m, J=3H), 4.22-4.08 (m, 2H), 3.77 (d, J=11.5 Hz, 1H), 3.66 (t, J=6.6 Hz, 2H), 3.30 (d, J=11.5 Hz, 1H), 2.70 (dd, J=15.7, 7.9 Hz, 1H), 2.56 (dd, J=15.7, 5.6 Hz, 1H), 2.28 (q, J=6.8 Hz, 2H), 2.08-2.18 (m, 1H), 1.57 (s, 3H), 1.46 (s, 9H), 1.06-0.92 (m, 11H), 0.83 (d, J=6.8 Hz, 3H), 0.76 (d, J=6.8 Hz, 3H), 0.03 (s, 9H). ¹³C NMR (101 MHZ, CDCl₃) δ 174.2, 170.3, 169.6, 161.9, 160.7, 139.3, 135.5, 135.5, 133.7, 133.7, 132.4, 129.6, 128.3, 127.6, 85.2, 71.9, 63.0, 56.8, 41.2, 39.7, 35.5, 31.1, 28.3, 26.8, 24.6, 19.1, 19.0, 17.4, 17.2, −1.6. HRMS (ESI): m/z calcd for C₄₇H₆₉N₄O₉SSi₂ (M+H)⁺: 921.4323, found: 921.4329.

(12Z,22Z,24R,5S,8S)-8-((E)-4-((tert-butyldiphenylsilyl)oxy)but-1-en-1-yl)-5-isopropyl-24-methyl-24,25-dihydro-7-oxa-4,11-diaza-1(4,2)-oxazola-2(2,4)-thiazolacyclododecaphane-3,6, 10-trione (20). Compound 20 was obtained using the same procedure for the preparation of compound 16, except that 19 was used instead of 15 and with elution by 5% CH₃OH in CH₂Cl₂, (19 mg, 25% yield). ¹H NMR (400 MHZ, CDCl₃) δ 7.95 (s, 1H), 7.65-7.58 (m, 4H), 7.45-7.30 (m, 6H), 7.05 (d, J=8.9 Hz, 1H), 6.15 (dd, J=9.7, 3.8 Hz, 1H), 5.87 (dt, J=14.5, 6.9 Hz, 1H), 5.57-5.66 (m, 1H), 5.49 (dd, J=15.6, 7.2 Hz, 1H), 4.92 (dd, J=17.6, 9.7 Hz, 1H), 4.50 (dd, J=8.9, 3.4 Hz, 1H), 4.03-3.90 (m, 2H), 3.67 (t, J=6.4 Hz, 2H), 3.21 (d, J=11.4 Hz, 1H), 2.85 (dd, J=16.6, 10.3 Hz, 1H), 2.61 (dd, J=16.6, 2.9 Hz, 1H), 2.33-2.19 (m, 2H), 2.00-2.12 (m, 1H), 1.79 (s, 3H), 1.01 (s, 9H), 0.65 (d, J=6.9 Hz, 3H), 0.61 (d, J=6.9 Hz, 3H). ¹³C NMR (101 MHZ, CDCl₃) δ 173.5, 170.7, 168.7, 163.3, 161.9, 141.2, 135.5, 135.0, 133.8, 133.8, 132.3, 129.6, 128.4, 127.7, 83.8, 72.2, 63.0, 58.1, 50.8, 43.3, 40.3, 37.2, 35.6, 34.2, 26.8, 26.8, 23.8, 23.5, 20.2, 19.2, 18.7, 17.2. HRMS (ESI): m/z calcd for C₃₇H₄₆N₄NaO₅SSi (M+Na)⁺: 725.2805, found: 725.2810.

(12Z,22Z,24R,5S,8S)-8-((E)-4-hydroxybut-1-en-1-yl)-5-isopropyl-24-methyl-24,25-dihydro-7-oxa-4,11-diaza-1(4,2)-oxazola-2(2,4)-thiazolacyclododecaphane-3,6,10-trione (21). Compound 21 was obtained using the same procedure for the preparation of compound 17, except that 20 was used instead of 16 and with elution by 5% CH₃OH in CH₂Cl₂, (8.8 mg, 89% yield). ¹H NMR (400 MHZ, CDCl₃) δ 7.97 (s, 1H), 7.54-7.46 (m, 1H), 7.06 (d, J=8.9 Hz, 1H), 5.85-5.76 (m, 1H), 5.69-5.54 (m, 2H), 4.82-4.65 (m, 1H), 4.56-4.34 (m, 1H), 4.10-3.85 (m, 2H), 3.72-3.46 (m, 2H), 3.24-3.18 (m, 1H), 2.98-2.74 (m, 2H), 2.60 (dd, J=15.8, 3.4 Hz, 1H), 2.25 (q, J=6.3 Hz, 2H), 2.06-1.91 (m, 1H), 1.77 (s, 3H), 0.66 (d, J=6.3 Hz, 3H), 0.61 (d, J=6.3 Hz, 3H). ¹³C NMR (101 MHZ, CDCl₃) δ 173.4, 173.4, 170.7, 168.7, 163.7, 141.3, 134.6, 131.4, 129.2, 83.3, 72.0, 60.9, 58.5, 42.9, 40.4, 37.2, 35.4, 33.8, 23.7, 18.6, 17.3. HRMS (ESI): m/z calcd for C₂₁H₂₈N₄NaO₅S (M+Na)³⁰ : 487.1627, found: 487.1623.

S-((E)-4-((12Z,22Z,24R,5S,8S)-5-isopropyl-24-methyl-3,6, 10-trioxo-24,25-dihydro-7-oxa-4, 11-diaza-1(4,2)-oxazola-2(2,4)- thiazolacyclododecaphane-8-yl)but-3-en-1-yl) octanethioate (3). (CAS Number: 1800584-53-5)⁴: Compound 3 was obtained using the same procedure for the preparation of compound 1, except that 21 was used instead of 10 and with elution by 5% CH₃OH in CH₂Cl₂ (6.3 mg, 60% yield). (eluted by 3% CH₃OH in CH₂Cl₂). ¹H NMR (400 MHZ, CDCl₃) 7.99 (s, 1H), 7.09 (d, J=9.0 Hz, 1H), 6.13 (dd, J=4.2, 8.6 Hz, 1H), 5.88-5.78 (m, 1H), 5.68-5.60 (m, 1H), 5.58-5.50 (m, 1H), 4.99 (dd, J=9.8, 17.6 Hz, 1H,), 4.55 (dd, J=3.5, 10.7 Hz, 1H), 4.10-3.97 (m, 2H), 3.25 (d, J=11.5 Hz, 1H), 2.95-2.83 (m, 3H), 2.69 (dd, J=2.9, 16.4 Hz, 1H), 2.53 (t, J=7.7 Hz, 2H), 2.32 (q, 2H), 2.17-2.03 (m, 1H), 1.85 (s, 3H), 1.72-1.57 (m, 2H), 1.38-1.18 (m, 8H), 0.88 (t, J=6.6 Hz, 3H), 0.69 (d, J=6.9 Hz, 3H), 0.65 (d, J=6.9 Hz, 3H). ¹³C NMR (101 MHZ, CDCl₃) δ 199.3, 173.4, 170.5, 168.7, 163.2, 161.8, 141.1, 135.0, 132.7, 128.4, 83.8, 71.9, 58.1, 44.1, 43.3, 40.3, 37.3, 34.2, 32.2, 31.6, 28.9, 27.9, 25.6, 23.8, 22.6, 18.7, 17.2, 14.1. HRMS (ESI): m/z calcd for C₂₉H₄₃N₄O₆S₂ (M+H)⁺: 607.2624, found: 607.2632. [α]²⁵_{D}: +19.9 (c=0.7, CH₂Cl₂), (lit. [α]²⁵_{D}: +18 (c=0.7, CH₂Cl₂)).

### REFERENCES

Each of the following references, or any other reference disclosed elsewhere herein, is hereby incorporated by reference in its entirety for all purposes.
1. Bowers, A. A.; Greshock, T. J.; West, N.; Estiu, G.; Schreiber, S. L.; Wiest, O.; Williams, R. M.; Bradner, J. E., J. Am. Chem. Soc., 2009, 131, 2900-2905.
2. Seiser, T.; Kamena, F.; Cramer, N., Angew. Chem., Int. Ed. 2008, 47, 6483-6485.
3. Chen, Q .-Y.; Chaturvedi, P. R.; Luesch, H. Org. Process, Res. & Dev., 2018, 22, 190-199.
4. Clausen, D. J.; Smith, W. B.; Haines, B. E.; Wiest, O.; Bradner, J. E.; Williams, R. M., Bioorg. & Med. Chem., 2015, 23, 5061-5074.
5. Guerra-Bubb, J. M.; Bowers, A. A.; Smith, W. B.; Paranal, R.; Estiu, G.; Wiest, O.; Bradner, J. E.; Williams, R. M., Bioorg. & Med. Chem. Lett., 2013, 23, 6025-6028.
6. Zhang, B.; Liu, J.; Gao, D.; Yu, X.; Wang, J.; Lei, X., Eur. J. Med. Chem., 2019, 182, 111672.
7. Akoto, D. O.; Rainier, J. D., Synthesis 2019; 51, 3529-3535

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred aspects, exemplary aspects and optional features, modification and variation of the concepts herein disclosed may be resorted to by those skilled in the art, and that such modifications and variations are considered to be within the scope of this invention as defined by the appended claims. The specific aspects provided herein are examples of useful aspects of the present invention and it will be apparent to one skilled in the art that the present invention may be carried out using a large number of variations of the devices, device components, methods steps set forth in the present description. As will be obvious to one of skill in the art, methods, and devices useful for the present methods can include a large number of optional composition and processing elements and steps.

When a group of substituents is disclosed herein, it is understood that all individual members of that group and all subgroups, including any isomers, enantiomers, and diastereomers of the group members, are disclosed separately. When a Markush group or other grouping is used herein, all individual members of the group and all combinations and subcombinations possible of the group are intended to be individually included in the disclosure. When a compound is described herein such that a particular isomer, enantiomer or diastereomer of the compound is not specified, for example, in a formula or in a chemical name, that description is intended to include each isomer and enantiomer of the compound described individual or in any combination. Additionally, unless otherwise specified, all isotopic variants of compounds disclosed herein are intended to be encompassed by the disclosure. For example, it will be understood that any one or more hydrogens in a molecule disclosed can be replaced with deuterium or tritium. Isotopic variants of a molecule are generally useful as standards in assays for the molecule and in chemical and biological research related to the molecule or its use. Methods for making such isotopic variants are known in the art. Specific names of compounds are intended to be exemplary, as it is known that one of ordinary skill in the art can name the same compounds differently.

Many of the molecules disclosed herein contain one or more ionizable groups groups from which a proton can be removed (e.g., —COOH) or added (e.g., amines) or which can be quaternized (e.g., amines). All possible ionic forms of such molecules and salts thereof are intended to be included individually in the disclosure herein. With regard to salts of the compounds herein, one of ordinary skill in the art can select from among a wide variety of available counterions those that are appropriate for preparation of salts of this invention for a given application. In specific applications, the selection of a given anion or cation for preparation of a salt may result in increased or decreased solubility of that salt.

Every formulation or combination of components described or exemplified herein can be used to practice the invention, unless otherwise stated.

Whenever a range is given in the specification, for example, a temperature range, a time range, a carbon atom range, or a composition or concentration range, all intermediate ranges, and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific aspects that are in the prior art. For example, when composition of matter is claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred aspects and optional features, modification and variation of the concepts herein disclosed may be resorted to by those skilled in the art, and that such modifications and variations are considered to be within the scope of this invention as defined by the appended claims.

We claim:

1. A method for preparing a compound of formula (III): 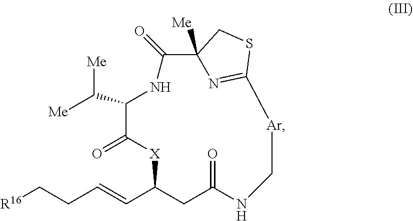
the method comprising:
reacting a compound of formula (I): 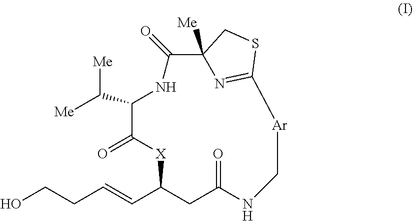
with an activating agent selected from C₁-C₂₀ linear or branched alkyl-sulfonyl halide, substituted alkyl-sulfonyl halide, arylsulfonyl halide, substituted arylsulfonyl halide, halo-phosphite, or a halogenating reagent, thereby forming a compound of formula (II): 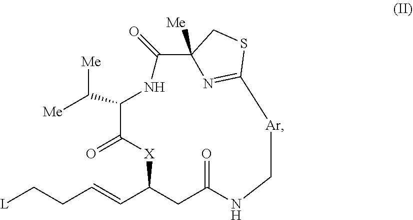
and reacting the compound of formula (II) with a nucleophile comprising an R¹ group, thereby forming the compound of formula (III);
wherein:
X is O or NH;
Ar is thiazolyl, pyridinyl, or oxazolyl;
R¹ is R₂COS, R₃S, or R⁴R₅N;
R², R³, R⁴, and R⁵independently are hydrogen, C₁-C₂₄ linear alkyl, C₁-C₂₄ linear alkyl substituted with one or more aryl or heteroaryl in any position, C₁-C₂₄ branched alkyl, C₁-C₂₄ branched alkyl substituted with one or more aryl or heteroaryl in any position, C₃-C₂₄ cyclic alkyl, C₃-C₂₄ cyclic alkyl substituted with one or more aryl or heteroaryl in any position, C₁-C₂₄ alkenyl, C₁-C₂₄ alkynyl, aryl, heteroaryl, diphenyl methyl, triphenylmethyl, or any substituted version thereof; and
L is a leaving group selected from C₁-C₂₄ linear or branched carbon sulfonate, substituted C₁-C₂₄ linear or branched sulfonate, arylsulfonate, substituted arylsulfonate, heteroarylsulfonate, substituted heteroarylsulfonate, phosphate, or halogen.

2. A method for preparing a compound of formula (I): 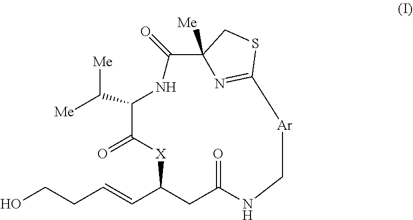
the method comprising reacting in one or more reactions:
a compound of formula (IV): 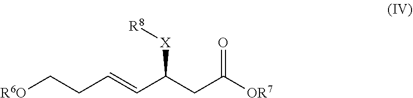
a compound of formula (V): 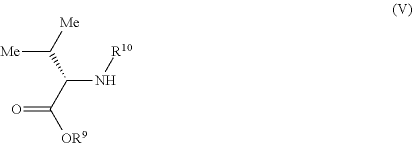
and a compound of formula (VI): 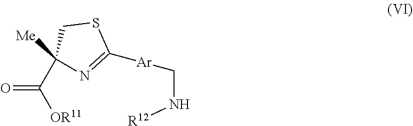
wherein:
X is O or NH;
Ar is thiazolyl, pyridinyl, or oxazolyl;
R⁸ is H or a suitable alcohol protecting group, R⁸, R¹⁰, and R¹² independently are H, a suitable amine protecting group, or a chiral group,
R⁷, R⁹, and R¹¹ independently are H, an alkali metal ion, an alkaline earth metal ion, or a suitable carboxylic acid protecting group;
optionally wherein the suitable carboxylic acid protecting group is selected from C₁-C₆ linear or branched alkyl, C₁-C₆ linear or branched alkyl substituted with one or more aryl or heteroaryl in any position, or alkylsilyl;
optionally wherein the C₁-C₆ linear or branched alkyl, or the C₁-C₆ linear or branched alkyl substituted with one or more aryl or heteroaryl in any position, is selected from methyl, ethyl, propyl, isopropyl, allyl, butyl, 2-butyl, tert-butyl, benzyl, or substituted benzyl; and
optionally wherein the alkylsilyl is 2-(trimethylsilyl)ethyl.

3. A method for preparing a compound of formula (III): 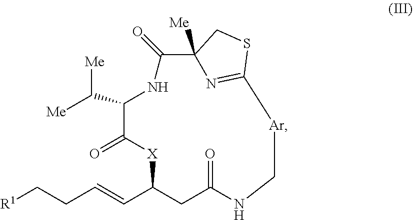
the method comprising:
reacting a compound of formula (II) with a nucleophile comprising an R¹ group: 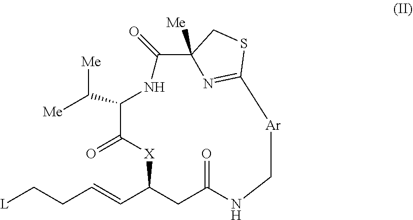
wherein:
X is O or NH;
Ar is thiazolyl, pyridinyl, or oxazolyl;
R¹ is R₂COS, R₃S, or R⁴R₅N;
R², R³, R⁴, and R⁵independently are hydrogen, C₁-C₂₄ linear alkyl, C₁-C₂₄ linear alkyl substituted with one or more aryl or heteroaryl in any position, C₁-C₂₄ branched alkyl, C₁-C₂₄ branched alkyl substituted with one or more aryl or heteroaryl in any position, C₃-C₂₄ cyclic alkyl, C₃-C₂₄ cyclic alkyl substituted with one or more aryl or heteroaryl in any position, C₁-C₂₄ alkenyl, C₁-C₂₄ alkynyl, aryl, heteroaryl, diphenyl methyl, triphenylmethyl, or any substituted version thereof; and
L is a leaving group selected from C₁-C₂₄ linear or branched carbon sulfonate, substituted C₁-C₂₄ linear or branched sulfonate, arylsulfonate, substituted arylsulfonate, heteroarylsulfonate, substituted heteroarylsulfonate, phosphate, or halogen.

4. The method of claim 3, further comprising producing the compound of formula (II) from a compound of formula (IX): 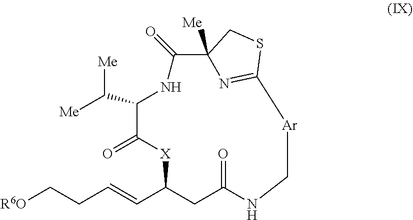
by converting OR⁶ of the compound of formula (IX) into the leaving group L;
wherein R⁶ is H or a suitable alcohol protecting group.

5. The method of claim 4, wherein converting OR⁶ into the leaving group L comprises:
when R⁶ is the suitable alcohol protecting group, deprotecting the suitable alcohol protecting group to provide the compound of formula (IX) where R⁶ is H; and
reacting the compound of formula (IX) where Re is H with an activating agent selected from C₁-C₂₀ linear or branched alkyl-sulfonyl halide, substituted alkyl-sulfonyl halide, arylsulfonyl halide, substituted arylsulfonyl halide, halo-phosphite, or a halogenating reagent.

6. The method of claim 4 or claim 5, wherein the compound of formula (IX) is a compound of formula (I): 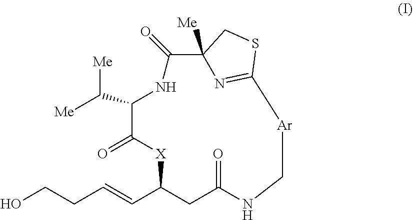
and the method comprises reacting the compound of formula (I) with an activating agent selected from C₁-C₂₀ linear or branched alkyl-sulfonyl halide, substituted alkyl-sulfonyl halide, arylsulfonyl halide, substituted arylsulfonyl halide, halo-phosphite, or a halogenating reagent, thereby forming the compound of formula (II).

7. The method of any preceding claim, wherein the compound of formula (I) or the compound of formula (IX) is a compound of formula (I-a), (I-b), or (I-c):

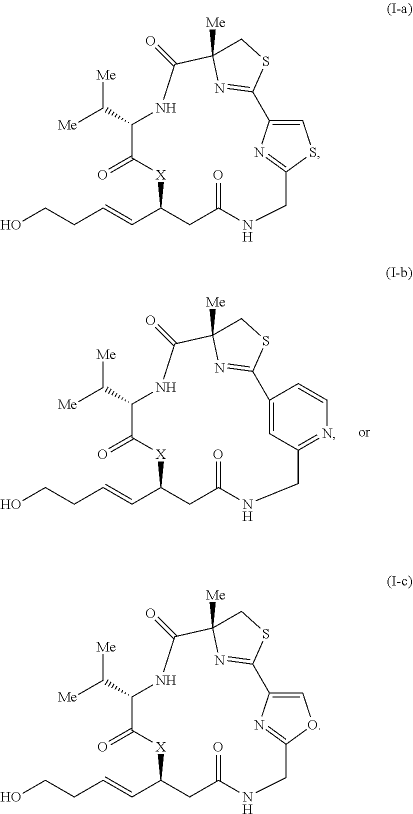

8. The method of any preceding claim, wherein L is OMs, OTs, OTf, 2,2,2-trifluoroethanesulfonate, n-octanesulfonate, benzenesulfonate, OP(═O)(OMe)₂, OP(═O)(OEt)₂, OP(═O)(OPh)₂, I, Br, Cl or F.

9. The method of any preceding claim, wherein Ar is:

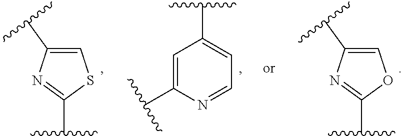 [SMILES: *c1csc(*)n1 ; *c1ccnc(*)c1 ; *c1coc(*)n1]

10. The method of any preceding claim, wherein the nucleophile is thiooctanoic acid, such that the compound of formula (III) is a compound of formula (XXV):

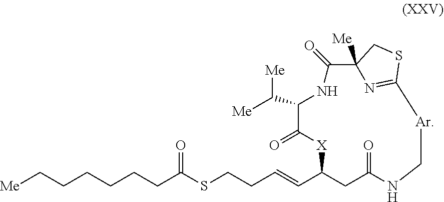

11. The method of claim 10, wherein X is NH and Ar is: 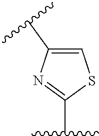 [SMILES: *c1csc(*)n1]
such that the compound of formula (III) is a compound of formula (1):

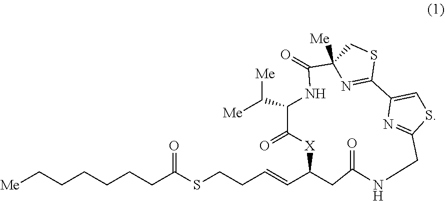

12. The method of claim 10, wherein X is O and Ar is: 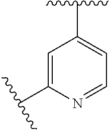 [SMILES: *c1ccnc(*)c1]
such that the compound of formula (III) is a compound of formula (2):

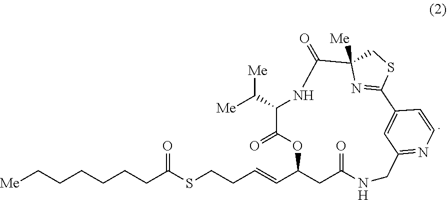 [SMILES: CCCCCCCC(=O)SCC/C=C/[C@@H]1CC(=O)NCc2cc(ccn2)C2=N[C@@](C)(CS2)C(=O)N[C@@H](C(C)C)C(=O)O1]

13. The method of claim 10, wherein X is O and Ar is: 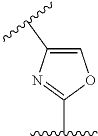 [SMILES: *c1coc(*)n1]
such that the compound of formula (III) is a compound of formula (3):

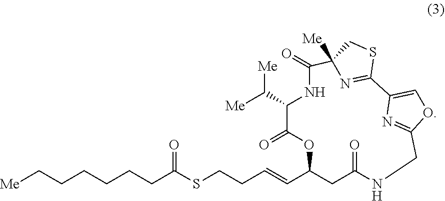 [SMILES: CCCCCCCC(=O)SCC/C=C/[C@@H]1CC(=O)NCc2nc(co2)C2=N[C@@](C)(CS2)C(=O)N[C@@H](C(C)C)C(=O)O1]

14. The method of any one of claim 1 or 4-13, wherein the method further comprises producing the compound of formula (I) or the compound of formula (IX) from one or more reactions comprising:
a compound of formula (IV): 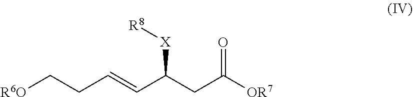
a compound of formula (V): 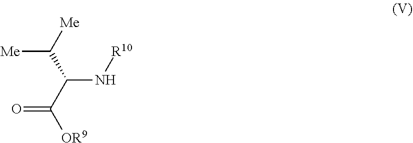
and a compound of formula (VI): 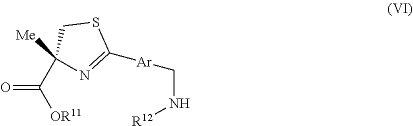
wherein:
R⁶ is H or a suitable alcohol protecting group,
R⁸, R¹⁰, and R¹² independently are H, a suitable amine protecting group, or a chiral group,
R⁷, R⁹, and R¹¹ independently are H, an alkali metal ion, an alkaline earth metal ion, or a suitable carboxylic acid protecting group;
optionally wherein the suitable carboxylic acid protecting group is selected from C₁-C₆ linear or branched alkyl, C₁-C₆ linear or branched alkyl substituted with one or more aryl or heteroaryl in any position, or alkylsilyl;
optionally wherein the C₁-C₆ linear or branched alkyl, or the C₁-C₆ linear or branched alkyl substituted with one or more aryl or heteroaryl in any position, is selected from methyl, ethyl, propyl, isopropyl, allyl, butyl, 2-butyl, tert-butyl, benzyl, or substituted benzyl; and
optionally wherein the alkylsilyl is 2-(trimethylsilyl)ethyl.

15. The method of claim 2 or claim 14, wherein:
the compound of formula (IV) is reacted with the compound of formula (V) via condensation to form a compound of formula (VII): 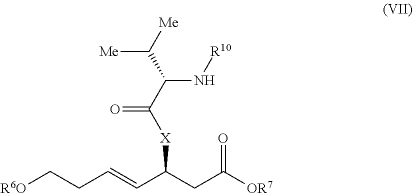
the compound of formula (VII) is reacted with the compound of formula (VI) via amidation to form a compound of formula (VIII): 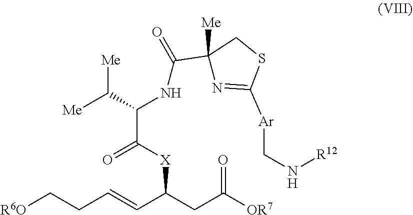
and the compound of formula (VIII) is reacted by amidating ring closure to form the compound of formula (IX) or the compound of formula (I) where R⁶ is H.

16. The method of claim 15, wherein:
R⁶ is H or a suitable alcohol protecting group;
in reacting the compound of formula (IV) with the compound of formula (V): R⁸ is H, and when X is N, the N is neutral or positively charged; R⁷ is a suitable carboxylic acid protecting group; R¹⁰ is a suitable amine protecting group; and R⁹ is H, an alkali metal ion, or an alkaline earth metal ion;
in reacting the compound of formula (VI) with the compound of formula (VII): R⁷ is a suitable carboxylic acid protecting group; R¹⁰ is H, and optionally R¹⁰ is attached to a positively charged nitrogen atom; R¹¹ is H, an alkali metal ion, or an alkaline earth metal ion; and R¹² is a suitable amine protecting group; and
in reacting the compound of formula (VIII) by amidating ring closure: Re is H or a suitable alcohol protecting group; R⁷ is H, an alkali metal ion, or an alkaline earth metal ion; and
R¹² is H, and optionally R¹² is attached to a positively charged nitrogen atom.

17. The method of any one of claim 2 or 14-16, wherein X is NH, and the method further comprises producing the compound of formula (IV) by:
reacting via imine formation a compound of formula (X): 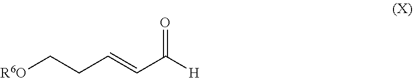
with a chiral auxiliary of formula (XI):
Y—NH₂ (XI)
to form a compound of formula (XII): 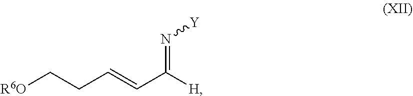
reacting the compound of formula (XII) via nucleophilic addition with a compound of formula (XIII): 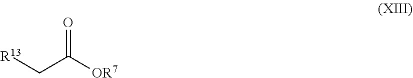
to form the compound of formula (IV) where X is NH and R⁸ is chiral group Y: 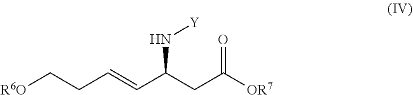
and cleaving the N—Y bond to form the compound of formula (IV) where R⁸ is H, and when X is N, the N is neutral or positively charged,
wherein:
Y is a chiral group sufficient to effect a diastereoselective nucleophilic addition of the compound of formula (XIII) to the compound formula (XII) to form the compound of formula (IV) as a majority product where R⁸ is chiral group Y,
R⁶ is a suitable alcohol protecting group,
R⁷ is a suitable carboxylic acid protecting group;
optionally wherein the carboxylic acid protecting group is selected from C₁-C₆ linear or branched alkyl, C₁-C₆ linear or branched alkyl substituted with one or more aryl or heteroaryl in any position, or alkylsilyl;
optionally wherein the C₁-C₆ linear or branched alkyl, or the C₁-C₆ linear or branched alkyl substituted with one or more aryl or heteroaryl in any position, is selected from methyl, ethyl, propyl, isopropyl, allyl, butyl, 2-butyl, tert-butyl, benzyl, or substituted benzyl; and
optionally wherein the alkylsilyl is 2-(trimethylsilyl)ethyl; and
R¹³ is H, a halide, or a metal comprising Li, Na, K, ZnBr, ZnCl, CuCl, CuBr, CuI, Cu, or any combination thereof.

18. The method of claim 17, wherein the chiral auxiliary of formula (XI) is: 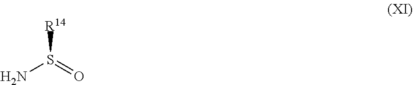
wherein R¹⁴ is tert-butyl, p-tolyl, alkyl, an aromatic group, or any substituted versions thereof.

19. The method of claim 18, wherein R¹⁴ is tert-butyl, such that the compound of formula (XI) is (R)-tert-butanesulfinamide.

20. A compound of formula (XXVI): 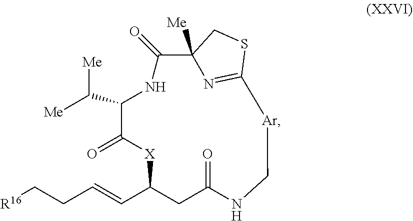
wherein:
X is O or NH;
Ar is thiazolyl, pyridinyl, or oxazolyl;
R₁₆ is OH, OR₈, or L;
R⁸ is a suitable alcohol protecting group; and
L is a leaving group selected from C₁-C₂₄ linear or branched carbon sulfonate, substituted C₁-C₂₄ linear or branched sulfonate, arylsulfonate, substituted arylsulfonate, heteroarylsulfonate, substituted heteroarylsulfonate, phosphate, or halogen.

21. The compound of claim 20, wherein the compound of formula (XXVI) is a compound of formula (I-a), (I-b), or (I-c):

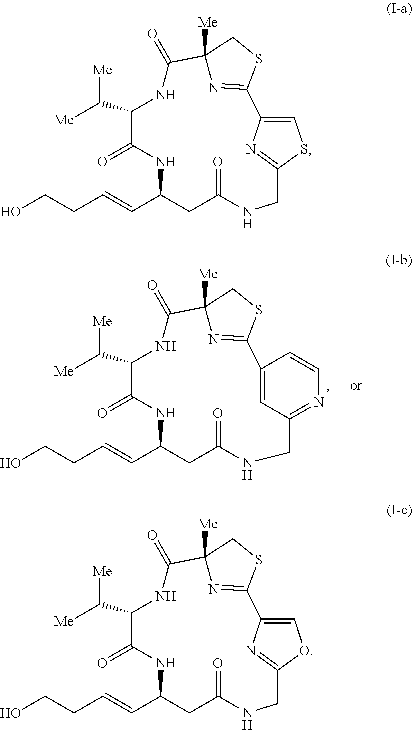 [SMILES: CC(C)[C@@H]1NC(=O)[C@]2(C)CSC(=N2)c2csc(n2)CNC(=O)C[C@@H](/C=C/CCO)NC1=O ; CC(C)[C@@H]1NC(=O)[C@]2(C)CSC(=N2)c2ccnc(c2)CNC(=O)C[C@@H](/C=C/CCO)NC1=O ; CC(C)[C@@H]1NC(=O)[C@]2(C)CSC(=N2)c2coc(n2)CNC(=O)C[C@@H](/C=C/CCO)NC1=O]

22. The compound of claim 20, wherein R₁₆ is OH.

23. The compound of claim 20, wherein R₁₆ is L.

24. The compound of claim 20, wherein R₁₆ is OR₆.

25. The compound of any one of claims 20-24, wherein:
X is O; and
Ar is pyridinyl.

26. The compound of any one of claims 20-24, wherein:
X is O; and
Ar is oxazolyl.

27. The compound of any one of claims 20-24, wherein:
X is NH; and
Ar is thiazolyl.
